(19) United States
(12) Patent Application Publication
(10) Pub. No.: US 2015/0247909 A1
(43) Pub. Date: Sep. 3, 2015

(54) Method and apparatus for determining a contrast agent enhancement

(71) Applicant: Martin Wawro, Dortmund (DE)

(72) Inventor: Martin Wawro, Dortmund (DE)

(73) Assignee: Digital Medics GmbH, Dortmund (DE)

(21) Appl. No.: 14/632,904

(22) Filed: Feb. 26, 2015

Related U.S. Application Data
(63) Continuation of application No. PCT/EP2013/067807, filed on Aug. 28, 2013.

(30) Foreign Application Priority Data
Aug. 28, 2012 (DE) 10 2012 107 926.9

(51) Int. Cl. G01R 33/56; A61B 5/055; G01R 33/48

(52) U.S. Cl. CPC G01R 33/5601; G01R 33/4818; A61B 5/055

(57) ABSTRACT

The present invention relates to a device and a method for determining a value of a relative contrast agent enhancement associated with a time sub-segment of a second time segment based on data of a subset of data from a first set of data and based on data of a subset of data from a second set of data.

### CROSS-REFERENCE TO RELATED PATENT APPLICATIONS

This patent application is a continuation of PCT/EP2013/067807, filed Aug. 28, 2013, which claims priority to German Application No. 10 2012 107 926.9, filed Aug. 28, 2012, the entire teachings and disclosure of which are incorporated herein by reference thereto.

### FIELD OF THE INVENTION

The subject matter relates to a method and an apparatus for determining a value of relative contrast agent enhancement in contrast-enhanced magnetic resonance imaging (MRI).

### BACKGROUND OF THE INVENTION

Over the past 20 years, magnetic resonance imaging (MRI) has become one of the most important non-invasive imaging methods in diagnostic radiology. The principle of MRI is based on the transmission of a sequence of transient radio frequency pulses (RF pulses) 110 to an object (for example, a human or an animal) with the coordinate system x=(x,y,z) which is placed in a strong (for example >=0.5T) static magnetic field. The static field, also referred to as \vec{B}₀, field, ensures that the magnetic moments of the nuclei located in the object 120 (the moments are caused by their spins) are aligned along the field lines. By the irradiation of the aforementioned RF pulse 110 to the object 120 as exemplarily shown in FIG. 1a, the aligned magnetic moments are briefly deflected in order to then precess around the field lines of the \vec{B}₀ field with their magnetic moment in decreasing amplitude. The frequency of the precession is called Larmor frequency (f_{L}), and it is:

$${f}_{L} = - \frac{γ}{2 π} \vert {\vec{B}}_{0} \vert$$

wherein γ is the gyromagnetic constant. In order to align the spins within the given magnetic field, the transient RF pulses must be sent with the Larmor frequency. The precession of the spins in turn transmit an electromagnetic signal 130 which, by means of suitable antennae, is recorded and used for further processing, generally after an echo time T_{E}. The field strength of the radiated RF pulse determines the deflection or flip angle of the spins. This type of MRI is called a gradient echo sequence. Another type of recording technique is the so-called spin echo sequence in which, after the irradiation of the RF pulse at the time

$$\frac{{T}_{E}}{2} ,$$

a further RF pulse is irradiated which rotates the spins by 180°. In both cases, the antennae start recording the radio waves emitted from the object in response thereto after a time T_{E}. The recorded signal is then discretized and fed into a digital computer system for further processing.

In order to differentiate various chemical compositions (e.g. different types of tissue) in an MRI scan, use is made of the fact that the gyromagnetic constant γ is a function of a specific class of atomic nuclei. In general, the nucleus of the hydrogen atom (¹H proton) is used, as it is available in varying concentrations or chemical bonding in all tissues in the body. The concentration and/or chemical bonding of the hydrogen has an effect on the amplitude of the recorded signal as well as on the time required by the excited atoms to once again become aligned according to the static \vec{B}₀ field and thus no longer emit any radio waves. This time span is referred to as relaxation time, and it has two distinct components: the T₁ relaxation and the T₂ relaxation. The T₁ relaxation denotes the time required to reach approx. 63% of the magnetization in the direction of the \vec{B}₀ field, and the T₂ relaxation denotes the time span required to reach 33% of the initial transverse magnetization, orthogonally to the \vec{B}₀ field. This second relaxation time results from the lost of phase coherence of the precession by the irradiation of the RF pulse.

Assuming that the \vec{B}₀ field is (largely) homogeneous, the hydrogen protons in the entire object are deflected following the irradiation of the RF pulse 110 with the appropriate Larmor frequency, and then in turn emit radio waves. In order to produce a three-dimensional coding of the object, another (linear) magnetic field is required, the so-called gradient field \vec{G}=(Gₓ,G_{y},G_{z}). This is created before and after the irradiation of the RF pulse in order to thereby produce a spatially variable magnetic field \vec{B}(x) with:

\vec{B}(x)=\vec{B}₀+\vec{G}(x)

This means that the precession frequency varies with a spatial variation of:

$$Δ {f}_{L} ( \underline{x} ) = - \frac{γ}{2 π} \vec{G} ( \underline{x} )$$

Therefore, if the gradient field is present at the time when the RF pulse is sent, the RF pulse excites only the areas S_{E} of the object that fulfill the following condition:

$${S}_{E} = \{ s ( \underline{x} ) ∈ S \vert \vert \vec{B} ( \underline{x} ) \vert = \frac{{f}_{L}}{γ} \}$$

Accordingly, only signals from the area S_{E} are received by the antennae. In order to differentiate the signal in other directions in space, two further techniques are used: frequency encoding and phase encoding. In these techniques, the radio frequency waves 130 emitted by the excited object areas are recorded in sequences and arranged in a vector space referred to as k space. In case of a (nearly) two-dimensional excited area, the k space is two-dimensional. In case of volume sequence recordings, the k space is three-dimensional. The frequency encoding changes the gradient field after the initial RF impulse, so that during the recording phase, the gradient runs in a predetermined direction within a plane of the excited area S_{E}. This gradient field changes the frequency of the radio waves 130 radiated by S_{E}, as a function of the position along the frequency encoding direction. The signal 130 recorded this way is stored within the k space 140 for instance in a row or column; an example hereof is shown in FIG. 1a for an exemplary 2D recording. In order to encode the remaining second plane direction, RF pulses are sequentially sent into the object, with a corresponding gradient field in the other direction being applied briefly within the T_{E} Interval. This field ensures during its application time, similar to the frequency encoding, a change in frequency, and after switching off the field, the spins of the protons in the phase position are shifted along the gradient. After expiry of the T_{E} time, the antennae then start the with the recording while the gradient field is added for the frequency encoding. This process is repeated sequentially with an increasing phase gradient field, while the results are stored in the k space according to the remaining direction. The time span between the two phase encoding steps is called repetition time T_{R}.

MRI is not limited to two-dimensional images. There are various techniques which can be used to acquire 3D volume data with this method. One of these techniques consists in using two interleaved phase encoding steps. Now, the coordinates of the 3D k space are referred to as k=(k₁,k₂,k₃) and denoted k₁ as the frequency encoding direction, and k₂ and k₃ as the two phase encoding directions. Similar to the 2D method, the RF pulse excites the entire sub-volume here, and as a result, each row in the k space receives information from all hydrogen nuclei of the sub-volume.

The acquired k space data contains the signal response with regard to the amplitude, frequency and phase position during the acquisition, but does not form an image of the object being examined. Rather, the antennae measure the transversal magnetization of the precessing hydrogen nuclei, which is a 2D quantity. A preferred annotation for the magnetization of nuclei therefore uses complex numbers in the following form:

e^{-iy\vec{G}}^{ₓ}^{xT}

where T is the time span for the respective component of the gradient field \vec{G}. The overall signal thus recorded for the entire object is presented as:

U=∫∫∫_{S}_{_{E}}I(x, y, z)e^{iy\vec{G}xT}dx dy dz (1)

If one now sets k₁=γ\vec{G}ₓTₓ, k₂=γ\vec{G}_{y}T_{y} and k₃=γ\vec{G}_{z}T_{z}, the equation (1) for this example of a 3D case can now be written as follows:

U(k₁, k₂, k₃)=∫∫∫_{S}_{_{E}}I(x, y, z)e^{-ik}_{₁}^{xk}_{₂}^{yk}_{₃}^{z}dx dy dz

which is exactly the Fourier transformation F(l,m,n) of the signal density I(x,y,z).

If one now looks at an infinitesimally small sub-volume within the object, the signal density (or amplitude) recorded by the antennae will depend on the type of recording sequence. For a gradient echo sequence, the signal depth is given as:

$${I}_{GE} ( {T}_{E} , {T}_{R} , α ) ∼ ρ \frac{1 - {e}^{- {T}_{R} / {T}_{1}}}{1 - cos α \cdot {e}^{- {T}_{R} / {T}_{1}}} sin α \cdot {e}^{- {T}_{E} / {T}_{2}^{*}}$$

where a is the flip angle, the initial angle by which the RF pulse deflects the longitudinal axis in transversal direction. The parameter T₂⁵⁰⁰ denotes the relaxation time for the T₂ relaxation taking into account local inhomogeneities, and ρ denotes the proton density within the sub-volume. For spin echo sequences, the signal density is defined by:

I_{SE}(T_{E}, T_{R})˜ρ(1−e^{T}^{_{R}}^{/T}^{₁})e^{T}^{_{E}}^{/T}^{₂}

The dynamic contrast agent MRI is used in a variety of ways in medical imaging—from perfusion analysis to tumor detection. The principle behind this method consists of first acquiring the patient's images (2D) or volume data (3D) in native form (i.e. without the use of a contrast agent). Then, the patient is injected with a contrast agent which alters the relaxation time in the area surrounding the contrast agent particles in accordance with their concentration.

After the contrast agent has been injected, the same areas of the patient are again recorded in prescribed time intervals. The result of this procedure is a time-series in which the concentrations of the contrast agent in tissue and blood vessels vary depending on the time, and insights into the pharmacokinetics of the contrast agent and the diagnostic and therapeutic consequences associated therewith.

A significant problem in the procedure described above is the temporal resolution in the image acquisition. For technical reasons, it lies (far) below the resolution required in order to achieve a sufficiently adequate assessment of the pharmacokinetics of the contrast agent.

FIG. 1b shows an example of the development over time of a exemplary contrast agent enhancement in the patient's body 130 after injection of the contrast agent, wherein the intensity of the contrast agent enhancement is shown on the y-axis 150.

A 3D gradient echo sequence for MRI mammography requires, for example, 40-120s for a “time point” in the time series, i.e. for the recording of a complete set of data in the k space, wherein about 6-8 such sequences are needed for a complete acquisition. A physically correct modelling of the pharmacokinetics requires knowledge of an arterial input function (AIF) which is determined essentially from the contrast agent concentration in a large arterial vessel at various points in time. Compared to the resolution of 3-5s required for an accurate measurement of an AIF, the technical resolution of 40-120 s is too low by at least one order of magnitude.

Based on the disadvantages described above, the subject matter had the object of providing a method and an apparatus with an improved estimation of a contrast agent concentration.

### SUMMARY OF THE INVENTION

This object is achieved in practice through a method which comprises determining a value of a relative contrast agent enhancement associated with a time sub-segment of a second time segment based on a subset of data from a first set of data and based on a subset of data from a second set of data, wherein the first set of data contains data from a multi-dimensional k space of a magnetic resonance image recorded without the influence of a contrast agent and was recorded in a first time segment, and wherein the second set of data contains data from a multi-dimensional k space of a magnetic resonance image recorded with the influence of a contrast agent and was recorded in a second time segment, which is different from the first time segment, wherein the data of the subset of data from the second set of data is associated with the time sub-segment of the second time segment and the data of the subset of data from the first set of data is associated essentially with the same region in the k space as the data of the subset of data from the second set of data.

This object is achieved in practice through a computer program product for determining a value of a relative contrast agent enhancement associated with a time sub-segment of a second time segment based on a subset of data from a first set of data and based on a subset of data from a second set of data, wherein the computer program product comprises a program for executing the method.

This object is achieved in practice through an apparatus comprising means for executing the method, in particular means for determining a value of a relative contrast agent enhancement associated with a time sub-segment of a second time segment based on a subset of data from a first set of data and based on a subset of data from a second set of data, wherein the first set of data contains data from a multi-dimensional k space of a magnetic resonance image recorded without the influence of a contrast agent and was recorded in a first time segment, and wherein the second set of data contains data from a multi-dimensional k space of a magnetic resonance image recorded with the influence of a contrast agent and was recorded in a second time segment, which is different from the first time segment, wherein the data of the subset of data from the second set of data is associated with the time sub-segment of the second time segment and the data of the subset of data from the first set of data is associated essentially with the same region in the k space as the data of the subset of data from the second set of data.

Moreover, this object is achieved in practice through an apparatus comprising at least one processor, at least one memory containing computer program code, wherein the at least one memory and the computer program code are set up, together with the at least one processor, to at least perform the method described above.

The method can, for example, comprise selecting the subset of data from the first set of data, and it can also comprise selecting the subset of data from the second set of data.

The multi-dimensional k space can be, for example, a two-dimensional k space corresponding to a two-dimensional MRI scan, wherein the set of data describes a two-dimensional image, or the multi-dimensional k space can also be, for example, a three-dimensional k space corresponding to a three-dimensional MRI scan, wherein the set of data describes a three-dimensional image and thus is able to represent a volume image set of data. The first set of data and/or the second set of data can be a raw set of data from an MRI scan.

For example, a set of data recorded during an MRI recording in the corresponding k space can be represented as a set of data Uₙ^{α}, wherein the index n stands for the number of the recorded set of data, i.e. for example for the association that the recorded set of data represents the nth set of data, and wherein the optional index α can specify the respective antenna α∈{1, . . . , A} from a number of A antennae, i.e. at least one antenna. Every nth set of data Uₙ^{α} is thus associated with a time segment in which the data of the respective set of data Uₙ^{α} was recorded.

The data of an acquired set of data Uₙ^{α} is therefore associated with a time segment in which the recording of the set of data Uₙ^{α} took place while running through the individual frequency steps and phase steps in the k space.

Thus this time segment can be, for example in a three-dimensional gradient echo sequence for an MRI mammography, between about 40 s and 120 s long, wherein the time segment may also have values deviating here from.

For example, a set of data Uₙ^{α} can comprise a total of m data values in the k space which may be successive, and which are, for example, recorded during the MRI recording by sampling:

Uₙ^{α}=(u_{n,1}^{α}, . . . , u_{n,m}^{α}) (2)

For example, a respective data value u_{n,l}^{α} (with l∈{1, . . . , m}) of the plurality of data values Uₙ^{α}=(u_{n,1}^{α}, . . . , u_{n,m}^{α}) can be associated with exactly one phase step and exactly one frequency step. Furthermore, a respective data value u_{n,l}^{α}, of the plurality of data values Uₙ^{α}=(u_{n,1}^{α}, . . . , u_{n,m}^{α}) can, for example, be associated with exactly one recording time within the time segment of the set of data with l∈{1, . . . , m}.

Depending on the MRI method used, it is also possible, for example, for several different data values u_{n,l}^{α} of the plurality of data values U_{n,l}^{α}=(u_{n,1}^{α}, . . . , u_{n,m}^{α}) to be associated with the same point in time, or substantially the same point in time. For example, data values u_{n,l}^{α} which are associated with the same phase step and respectively different frequency steps, can be associated with the same or substantially the same point in time, while data values u_{n,l}^{α} which are associated with different phase steps can each be associated with different points in time corresponding to the temporal occurrence of the respective phase step.

However, it is also possible that all data values u_{n,l}^{α} of a set of data Uₙ^{α} are each associated with different points in time within the time segment, wherein the time interval between the recorded data values may be the same, or wherein the time interval may, depending on the MRI method used, however also vary between different adjacent data values. Each of the data values is associated with one point in time within the time segment of the set of data Uₙ^{α}.

The representation of the data values of the set of data Uₙ^{α} may also deviate from (2), for example by a two or multi-dimensional matrix, in which the data values are deposited for example in columns and rows, i.e. a respective data value u_{n,l}^{α} (with l∈{1, . . . , m}) of the plurality of data values Uₙ^{α}=(u_{n,1}^{α}, . . . , u_{n,m}^{α}) can, for example, be associated with a column and a row. The plurality of data values Uₙ^{α}=(u_{n,1}^{α}, . . . , u_{n,m}^{α}) can therefore also be considered a special representation of a multi-dimensional k space, such as a two or three-dimensional k space.

The first set of data used, which was recorded without the influence of a contrast agent and which is associated with the first time segment, may, for example, be represented as U₁^{α}.

Furthermore, the second set of data, which was recorded with the influence of a contrast agent and which is associated with the second time segment, may, for example, be represented as U₂^{α}.

This second time segment can, for example, be situated in time after the first time segment. Thus this first set of data can be, for example, the MRI scan of a patient without the use of a contrast agent, wherein the patient is injected with a contrast agent following this MRI scan, and after the injection of the contrast agent, a second MRI scan is done of the same patient, preferably in the same position as during the first MRI scan, for the recording of the second set of data.

However, the second set of data can, for example, also be recorded prior to the first set of data, i.e. the first time segment can, for example, also be situated in time after the second time segment. Thus, a patient can, for example, be injected with a contrast agent first, and after the injection of the contrast agent, an MRI scan of the patient is performed to record the second set of data. Then one waits until the injected contrast agent no longer has any significant effect on an MRI scan, so that then another MRI scan of the same patient is performed, if possible in the same position, to record the first set of data. Therefore, the recording of the first set of data without the influence of a contrast agent can, for example, also be understood such that then the influence of the contrast agent is very low compared to the freshly injected contrast agent.

From the first set of data U₁^{α}, a subset of data can be selected, wherein the data of the subset is associated with a time sub-segment of the first time segment. The subset of data thus contains fewer data values than the entire first set of data U₁^{α}, i.e. the time sub-segment represents an excerpt of the first time segment. For example, the subset of data can comprise those data values u_{1,l}^{α} with l∈{1, . . . , m}, whose associated time points lie within the time sub-segment. For example, all data values from the first set of data U₁^{α} which lie within the time sub-segment can represent the subset of data, or a selection of data values from the totality of the data values of the first set of data which lie within the time sub-segment can represent the subset of data.

Thus, the subset of data can, for example, comprise a total of k data values of the first set of data U₁^{α} with k<m, whose associated time points each lie within the time sub-segment, wherein, for example, the selected data values can be specified through a quantity of k indices iₓ∈{1, . . . , m} with x∈{1, . . . , k}, so that, for example, a respective xth index iₓ is clearly associated with exactly one of the data values from the total of k data values from the subset of the first set of data U₁^{α}, wherein the data value u_{1,i}_{ₓ}^{α} corresponds to this data value. Therefore, the data values u_{1,i}_{₁}^{α}, . . . , u_{1,i}_{ₖ}^{α} represent the k data values of the subset of the first set of data U₁^{α}, wherein, for example, the selection of the data values of the first set of data U₁^{α} can be made by associating the k indices iₓ with the corresponding indices {1, . . . , m} in the k space.

Furthermore, a subset of data can, for example, be selected from the second set of data U₂^{α}, wherein the subset of data from the second set of data U₂^{α} contains fewer data values than the total second set of data U₂^{α}, i.e., the time sub-segment represents an excerpt of the second time segment. For example, the subset of data can comprise those data values u_{n,l}^{α} with l∈{1, . . . , m} whose time points lie within the time sub-segment of the second time segment. For example, all data values from the second set of data U₂^{α} which lie within the time sub-segment of the second time segment can represent the subset of data, or a selection of data values from the totality of the data values of the second set of data which lie within the time sub-segment of the second time segment, can be considered a subset of data.

The relative temporal position of the time sub-segment within the second time segment can therefore correspond to the relative temporal position of the time sub-segment within the first time segment, and the length of the time sub-segment of the second time segment can substantially or exactly correspond to the length of the time sub-segment of the first time segment.

The data of the subset of data from the first set of data U₁^{α} and the data of the subset of data from the second set of data U₂^{α} have the characteristic that the data of the subset of data from the first set of data U₁^{α} is associated with substantially the same region in the k space as the data of the subset of data from the second set of data U₂^{α}.

If, for example, a data value of the subset of data from the second set of data U₂^{α} represents the data value u_{2,l}^{α} with l∈{1, . . . , m}, then, for example, the data value of the subset of data from the first set of data located in the same position in the k space can represent the corresponding data value u_{1,l}^{α}, since the same index l represents the same point in the k space. Therefore, for example, the subset of data of the first set of data can have, for each data value u_{2,l}^{α} of the subset of data of the second set of data U₂^{α}, a data value u_{1,l}^{α} located, in each case, at the same, or substantially the same, point in the k space.

If the subset of data of the first set of data comprises, for example, a total of k data values of the first set of data U₁^{α} with k<m, with the data values u_{1,i}_{₁}^{α}, . . . , u_{1,i}_{ₖ}^{α} representing the k data values of the subset of the first set of data U₁^{α}, the data values of the subset of the second set of data U₂^{α} can, for example, be specified through the set of k indices iₓ∈{1, . . . , m} with x∈{1, . . . , k} already used for the subset of data of the first set of data, so that u_{2,i}_{₁}^{α}, . . . , u_{2,i}_{ₖ}^{α} represent the k data values of the second set of data U₂^{α}. This association can, for example, also occur in the reverse, i.e. if the data values u_{2,i}_{₁}^{α}, . . . , u_{2,i}_{ₖ}^{α} represent the k data values of the subset of the second set of data U₂^{α}, the data values of the subset of the first set of data U₁^{α} can, for example, be specified through the set of k indices iₓ∈{1, . . . , m} with x∈{1, . . . , k} already used for the subset of data of the second set of data, so that u_{1,i}_{₁}^{α}, . . . , u_{1,i}_{ₖ}^{α} represent the k data values of the first set of data U₁^{α}.

The data of the subset of data from the first set of data U₁^{α} comprises several data values and the data of the subset of data from the second set of data U₂^{α} comprises several data values. Thus, k≧2 may apply, and/or, if for example all data values from the first set of data U₁^{α} which lie in the time sub-segment represent the subset of data from the first set of data U₁^{α}, and if, for example, all data values from the second set of data U₂^{α} which lie in the time sub-segment of the second time segment represent the subset of data from the second set of data U₂^{α}, m≧2 may apply.

Then, a value of a relative contrast agent enhancement associated with the time sub-segment of the second time segment is calculated based on data of the subset of data from the first set of data U₁^{α} and on data of the subset of data from the second set of data U₂^{α}. Thus, for example, the several data values of the first subset of data from the first set of data U₁^{α} and the several data values of the second subset of data from the second set of data U₂^{α} can be used to determine the value of a relative contrast agent enhancement associated with the time sub-segment of the second time segment.

In this, a single value of a relative contrast agent enhancement is, for example, calculated for the time sub-segment of the second time segment, hereinafter always referred to as second time sub-segment, wherein the information is exploited that the MRI scan of the first set of data was done without contrast agent and the MRI scan of the second set of data under the influence of the contrast agent.

As the data used in determining the value of a relative contrast agent enhancement from the second set of data is that which was recorded during the second time sub-segment —i.e. data from the second set of data lying within the second time segment, but outside of the second time sub-segment, is not used—, the contrast agent enhancement present in the patient's body during this second time sub-segment is the primary factor influencing the determination of the value of a relative contrast agent enhancement.

Therefore, the present method allows for the temporal resolution of the determined value of a relative contrast agent enhancement to be improved compared to traditional methods which determine the value of a relative contrast agent enhancement based on all data acquired in the k space.

For example, by selecting the time sub-segment of the second time segment accordingly, the temporal resolution of the determined value of a relative contrast agent enhancement can be specified, for example simultaneously with a trade-off between the temporal resolution and the accuracy of the determined value of a relative contrast agent enhancement. If the selected time sub-segment of the second time segment is very short, the selected subset of data from the second set of data and the respectively selected subset of data from the first set of data is accordingly relatively small, i.e. only a few data values are used, so that the temporal resolution is high, but the accuracy of the determined value of a relative contrast agent enhancement is lower, compared to a value of a relative contrast agent enhancement determined based on a longer time sub-segment of the second time segment.

The determination of the value of a relative contrast agent enhancement associated with the second time sub-segment based on data of the subset of data from the first set of data and based on data of the subset of data from the second set of data can be done through various methods, for example through a suitable fitting method, in which a value of a relative contrast agent enhancement is determined, for example by determining, with such suitable fitting method, the value of a contrast agent enhancement which maps the data of the subset of data from the first set of data to the subset of data from the second set of data with sufficient accuracy, using the value of a contrast agent enhancement. For this, various fitting methods can be used, which, for example, can be based on the sum of the smallest error squares, or on other methods.

According to an advantageous embodiment, it is suggested that the method comprise the selection of the subset of data from the first set of data, and the selection of the subset of data from the second set of data.

Thus, for example, an apparatus and a method are described for determining a value of a relative contrast agent enhancement associated with a time sub-segment of a second time segment based on a subset of data from a first set of data and based on a subset of data from a second set of data, wherein the first set of data contains data from of a multi-dimensional k space of a magnetic resonance image recorded without the influence of a contrast agent and was recorded in a first time segment, and wherein the second set of data contains data from of a multi-dimensional k space of a magnetic resonance image recorded with the influence of a contrast agent and was recorded in a second time segment, which is different from the first time segment, wherein the data of the subset of data from the second set of data is associated with the time sub-segment of the second time segment and the data of the subset of data from the first set of data is associated essentially with the same region in the k space as the data of the subset of data from the second set of data.

According to an advantageous embodiment, it is suggested that the multi-dimensional k space be frequency-encoded in a first dimension with a plurality of frequencies and phase-coded in at least one additional dimension with a plurality of phase steps.

Thus, the k space can, for example, represent a two-dimensional k space, wherein a first direction in the k space is frequency-encoded with a plurality of frequencies in accordance with the first dimension, and wherein a second direction in the k space, which is preferably orthogonal to the first direction, is encoded with a plurality of phase steps in accordance with the second dimension.

The k space can, for example, also represent a three-dimensional k space, wherein a first direction in the k space is frequency-encoded with a plurality of frequencies in accordance with the first dimension, and wherein a second direction in the k space, which is preferably orthogonal to the first direction, is encoded with a plurality of phase steps in accordance with the second dimension, and wherein a third direction in the k space, which is preferably orthogonal to the first and the second direction, is encoded with a plurality of phase steps in accordance with a third dimension. In this, the phase steps of the plurality of phase steps of the second dimension are distinct, for example, from the phase steps of the third dimension.

Thus, the phase steps of the plurality of phase steps of the second dimension can, for example, be interleaved with the phase steps of the plurality of phase steps of the third dimension. However, it is also possible to use other techniques with regard to the specification of the phase steps of the plurality of phase steps of the second Dimension and the specification of the phase steps of the plurality of phase steps of the third dimension for scanning in the three-dimensional k space.

During the MRI scan, for example, the process runs through the respective phase encoding steps of the at least one additional dimension in the k space, and while this occurs, a set of data Uₙ^{α}, can be recorded in the corresponding k space.

For example, the recording of a set of data Uₙ^{α} in the k space can take place such that the recording starts with a phase step from the plurality of phase steps of one dimension of the at least one additional dimension and for this selected phase step, the accordingly associated data in the k space is recorded, wherein the data recorded for this selected phase step is associated in each case with the various frequencies of the plurality of frequencies. Then it is possible to continue with a further phase step from the plurality of phase steps of one dimension of the at least one additional dimension, wherein, in turn, the data associated accordingly in the k space is recorded for this selected phase step, wherein the data recorded for this selected phase step is, in turn, associated with the various frequencies of the plurality of frequencies, for example via a recording index of the signal received by the receiving antenna. Thus, it is possible to run through each phase step of the plurality of phase steps of one dimension of the at least one additional dimension, wherein the data recorded for this selected phase step is associated with different frequencies of the plurality of frequencies, so that, for example, a set of data of a two-dimensional or three-dimensional k space can be recorded. In the two-dimensional k space, the at least one additional dimension comprises, for example, exactly one additional dimension which is phase-encoded with a plurality of phase steps.

In the three-dimensional k space, the at least one additional dimension comprises, for example, exactly two additional dimensions, wherein a first additional dimension of the two additional dimensions is phase-encoded with a first plurality of phase steps, and a second additional dimension of the two additional dimensions is phase-encoded with a second plurality of phase steps. In this three-dimensional case, a first phase step from the first plurality of phase steps and a second phase step from the second plurality of phase steps is used for phase encoding in the two additional dimensions during an MRI scan, wherein the data then recorded in the k space for this first phase step of the first additional dimension and for this phase step of the second additional dimension, are in each case associated in turn with the various frequencies of the plurality of frequencies. Thus a complete 3D scan can be generated for example by combining all phase steps of the first plurality of phase steps respectively with each phase step of the second plurality of phase steps into a pair of phase steps, and by recording the data for each combination in the k space, which data in turn is associated with the various frequencies of the plurality of frequencies.

Accordingly, one phase step of the first plurality of phase steps for phase encoding in the first additional dimension and one phase step of the second plurality of phase steps for phase encoding in the second additional dimension are used in the 3D scan, wherein the phase encoding in the first additional dimension and the phase encoding in the second additional dimension occurs simultaneously. This can be implemented, for example, by applying, in the spatial direction of the first additional dimension, a gradient field in accordance with the phase encoding of the first additional dimension and, simultaneously, in the spatial direction of the second additional dimension, a gradient field in accordance with the phase encoding of the second additional dimension, and switching these gradient fields off again prior to the reading of the received signal. For example, the X direction of a space can represent the first dimension which is frequency-encoded, wherein X direction of the space can be the first additional dimension and the Y direction of the space can be second additional dimension, wherein two gradient fields in Y and Z direction are modulated simultaneously, wherein the strength of the field of the respective gradient field of the two gradient fields steers the respective phase encoding in the corresponding direction, i.e. the corresponding further dimension.

The data of an acquired set of data Uₙ^{α} is therefore associated with a time segment in which the recording of the set of data Uₙ^{α} took place while running through the individual phase steps or combination of pairs of phase steps of the first and second additional dimension in the k space.

For example, in the 2D case, a respective data value u_{n,l} ^{α} (with l∈{1, . . . , m}) of the plurality of data values Uₙ^{α}=(u_{n,1}^{α}, . . . , u_{n,m}^{α}) can be associated with exactly one phase step and exactly one frequency, or in the 3D case, a respective data value u_{n,l}^{α} (with l∈{1, . . . , m}) of the plurality of data values Uₙ^{α}=(u_{n,1}^{α}, . . . , u_{n,m}^{α}) can be associated, for example, with exactly one phase step from the first plurality of phase steps, exactly one second phase step from the second plurality of phase steps, i.e. a pair of phase steps of the first and second additional dimension, and exactly one frequency. Furthermore, a respective data value u_{n,l}^{α} of the plurality of data values Uₙ^{α}=(u_{n,1}^{α}, . . . , u_{n,m}^{α}) can, for example, be associated with exactly one recording time within the time segment of the set of data with l∈{1, . . . , m}.

Depending on the MRI method used, it is also possible, for example, for several different data values u_{n,l}^{α} of the plurality of data values Uₙ^{α}=(u_{n,1}^{α}, . . . , u_{n,m}^{α}) to be associated with the same point in time, or substantially the same point in time. For example, data values u_{n,l}^{α}, which are associated with the same phase step or the same pair of phase steps and, in each case, different frequencies, can be associated with the same or substantially the same point in time, while data values u_{n,l}^{α}, which are associated with different phase steps or different pairs of phase steps, can be associated, in each case, with different points in time, in accordance with the temporal occurrence of the respective phase step or the respective pair of phase steps.

However, it is also possible that all data values u_{n,l}^{α} of a set of data Uₙ^{α} are each associated with different points in time within the time segment, wherein the time interval between the recorded data values may be the same, or wherein the time interval may, depending on the MRI method used, however also vary between different adjacent data values. Each of the data values is associated with one point in time within the time segment of the set of data Uₙ^{α}.

According to an advantageous embodiment, it is suggested that the time sub-segment of the second time segment be selected such that the data of the subset of data from the second set of data in one dimension of the at least one additional dimension is associated with one of the following: exactly one phase step of the plurality of phase steps of the dimension, and several successive phase steps of the plurality of phase steps of the dimension.

This it is possible to select exactly one phase step or several successive phase steps from the plurality of phase steps, and based on this selected exactly one phase step or the selected several successive phase steps, corresponding data values can be selected from the second set of data, i.e. data values, which are associated with the selected first phase step or the selected several successive phase steps, wherein the selected data values are associated with the subset of data from the second set of data.

The respectively selected data values of the subset of data from the second set of data are therefore associated with a time sub-segment of the second time segment, which depends on the selected exactly one phase step or the selected several successive phase steps.

In the 3D case, the selected phase step or the several successive phase steps is, for example, associated with the first additional dimension, while simultaneously, one phase step or several successive phase steps from the second plurality of phase steps is/are selected, so that the respectively selected data values of the subset of data from the second set of data are therefore associated with a time sub-segment of the second time segment which is dependent on the selected exactly one phase step or the selected several successive phase steps of the first additional dimension and on the selected exactly one phase step or the selected several successive phase steps of the second additional dimension.

According to an advantageous embodiment, it is suggested that the time sub-segment of the second time segment be determined based on the acquisition parameters of the exactly one phase step or the several successive phase steps, the echo time and the repetition time.

For example, the point in time of the recording T(s) can be determined at the start of each phase step pₛ (or each pair of phase steps) of a recording of an nth set of data Uₙ^{α} as follows:

T(s,n)=(s−1)T_{R}+T_{E}+tₙ

Thus, T_{R} represents, for example, the repetition time between two adjacent phase steps, T_{E} represents, for example, the time after which the antennae will start recording, and tₙ represents, for example, the point in time when the first phase step s=1 of the nth set of data Uₙ^{α} starts.

For example, the time of recording of the data associated with the various frequencies during a phase step of the plurality of phase steps may be negligibly short, so that the recorded data values for this one phase step pₛ and the various frequencies associated with this phase step can be associated, at least approximately, with the same point in time T(s).

If the time sub-segment of the second time segment is to be a relatively short time segment, i.e. if the temporal resolution of the determined value of a relative contrast agent enhancement is to be relatively small, it is possible to preferably select only a single phase step or in the 3D case, only a single pair of phase steps, so that all data values of the subset of data of the first set of data and of the subset of data of the second set of data are associated solely with this one selected phase step or selected pair of phase steps.

If, therefore, the data values of the subset of data of the second set of data are associated with exactly one phase step pₛ, wherein this phase step pₛ can also be, for example, a phase step of a pair of phase steps, the corresponding time sub-segment of the second time segment lies, for example, within the time segment between T(s,n) and T(s+1,n), and if the frequency steps for this phase step pₛ are run through in a very short time, the time sub-segment can approximately be seen as a very short time segment which starts at T(s,n) and ends very shortly thereafter, i.e. the time sub-segment of the second time segment can be considered an approximate point in time.

If the time sub-segment of the second time segment is to represent, for example, a somewhat longer time segment, i.e. if the temporal resolution of the determined value of a relative contrast agent enhancement is to be higher than in the aforementioned example, in which only one phase step is selected, it is possible to preferably select several phase steps of the plurality of phase steps, wherein these several phase steps are preferably directly adjacent, so that the selected subset of data of the first set of data and the subset of data of the second set of data are associated with these several selected phase steps. In the 3D case, these several phase steps of the plurality of phase steps can be the selected phase steps of the first plurality of phase steps of the first additional dimension, while simultaneously, exactly one phase step or several adjacent phase steps of the second plurality of phase steps can be selected, so that corresponding pairs of phase steps from the several phase steps of the first plurality of phase steps and from the exactly one phase step or the several phase steps of the second plurality of phase steps are formed, so that the selected subset of data of the first set of data and of the subset of data of the second set of data are associated with these pairs of phase steps.

Furthermore, if between the individual frequencies, there is a significant time segment which is defined, for example, by T_{f}, the point in time for a certain phase step pₛ (or a pair of phase steps) and a certain frequency fⱼ. can be determined, for example, as follows:

T(s, j, n)=(s−1)T_{R}+(j−1)T_{f}+T_{E}tₙ

According to an advantageous embodiment, it is suggested that the data of the subset of data from the second set of data in the first dimension be associated with one of the following: exactly one frequency step of the plurality of frequencies, a subset of several frequencies from of the plurality of frequencies, and all frequency steps of the plurality of frequencies.

For example, the data of the subset of data from the second set of data can be determined such that initially a phase step pₛ with s∈{1, . . . , w} of the plurality of w phase steps p₁, . . . , p_{w} or, in the 3D case, a pair of phase steps p_{s,q} with s∈{1, . . . , w} of the first plurality of w phase steps p₁, . . . , p_{w} and q of the second plurality of w₂ phase steps p′₂, . . . , p′_{w}_{₂} is selected, wherein this selected phase step pₛ corresponds, for example, to the previously described exactly one phase step or is selected from the several successive phase steps, or, in the 3D case, the selected pair of phase steps p_{s,q} is selected from the set of pairs of phase steps, and wherein for this selected phase step pₛ or for each pair of phase steps p_{s,q} for each frequency fⱼ. with j∈{1, . . . , v} of the exactly one frequency, or of the subset of several frequencies, or all frequencies of the plurality of v frequencies f₁, . . . , fᵥ one data value u_{2,l}^{α} of the second set of data U₂^{α} is selected in each case, which is associated with the selected phase step pₛ or the selected pair of phase steps p_{s,q} and the respective frequency step fⱼ, and which is associated with the subset of data from the second set of data, and wherein the index l denotes the corresponding point in the k space as a function of fⱼ and pₛ or p_{s,q}.

For each of the selected data values u_{2,l}^{α} of the second set of data U₂^{α}, a corresponding data value u_{1,l}^{α} of the first set of data U₁^{α} can be selected, wherein this data value u_{1,l}^{α} of the first set of data U₁^{α} is likewise associated with the selected phase step pₛ or the selected pair of phase steps p_{s,q} and the respective frequency step fⱼ, and which is associated with the subset of data from the first set of data.

This can be carried out, for example, for each of the phase steps of the exactly one phase step or of the several successive phase steps or for each pair of phase steps, so that subsequently, the data of the subset of data from the second set of data is selected and, similarly, the corresponding data of the subset of data from the first set of data can be selected.

According to an advantageous embodiment, it is suggested that the value of a relative contrast agent enhancement associated with the time sub-segment of the second time segment be determined based on the differential values between the data of the subset of the second set of data and the data of the subset of the first set of data, wherein a respective differential value of the differential values is formed based on the difference between one data value of the data of the subset of the second subset and the data value of the data of the subset of the first set of data associated with said data value.

Thus, for example, the differential values between data of the subset of the second set of data U₂^{α} and data of the subset of the first set of data U₁^{α} can be formed, wherein a respective differential value dₗ^{α} based on the difference a data value u_{2,l}^{α} of the data of the subset of the second set of data U₂^{α} and of the data value u_{1,l}^{α} of the data of the subset of the first set of data U₁^{α} associated with said data value u_{2,l}^{α} in the k space, for example by

dₗ^{α}=u_{2,l}^{α}−u_{1,l}^{α}.

If, for example, the subset of the first set of data U₁^{α} and the subset of the second set of data U₂^{α} includes, respectively, k selected data values and if the previously explained k indices iₓ∈{1, . . . , m} are used, an xth differential value (with x∈{1, . . . , k}) of the in total k differential values can be calculated as follows:

dᵢ_{ₓ}^{α}=u_{2,i}_{ₓ}^{α}−u_{1,i}_{ₓ}^{α}.

The respective data value u_{1,i}_{ₓ}^{α} of the data of the subset of the first set of data U₁^{α} contains, for example, the signal response in terms of amplitude, frequency and phase position during the acquisition in the k space associated with the data value u_{2,i}_{ₓ}^{α}, wherein the signal response does not comprise any portion caused by a contrast agent. In comparison to the respective data value u_{1,i}_{ₓ}^{α} of the data of the subset of the first set of data U₂^{α}, the associated differential value u_{2,i}_{ₓ}^{α} of the data of the subset of the second set of data U₂^{α} contains an additional signal component Δₗ^{α}, which is, for example, proportional to the relative contrast agent enhancement or represents a value of a relative contrast agent enhancement:

u_{2,i}_{ₓ}^{α}=u_{1,i}_{ₓ}^{α}+Δᵢ_{ₓ}^{α}

This additional signal component Δᵢ_{ₓ}^{α} can be determined, for example, by forming the differential value dᵢ_{ₓ}^{α}, and can be seen, for example, as an estimation of the relative contrast agent enhancement for the point in the k space associated with the differential value dᵢ_{ₓ}^{α}.

Each of the determined differential values dᵢ_{ₓ}^{α} can therefore be considered, for example, an estimation of the relative contrast agent enhancement in the respective point in the k space, so that it is possible to determine, for example based on an averaging of the determined differential values (or of the amounts of the determined differential values), the value of a relative contrast agent enhancement associated with the second time sub-segment. If only a single differential value is determined, this differential value or the amount of this differential value can be seen, for example, as the value of a relative contrast agent enhancement associated with the second time sub-segment.

According to an advantageous embodiment, it is suggested that a metric be calculated based on the determined differential values, wherein the metric represents a measure for the deviation between the differential values and a value of a relative contrast agent enhancement.

According to an advantageous embodiment, it is suggested that the calculation of the metric for each of the differential values includes the calculation of a differential value of the metric, which represents the difference between the respective differential value and a value of a relative contrast agent enhancement, and the calculation of the metric includes the calculation of the deviation measure on the basis of the calculated differential values of the metric.

For example, the metric can be calculated by calculating a differential value of the metric m′ₗ^{α} for each or for a selection of the determined differential values dₗ^{α}, which is calculated based on the difference between the respective differential value dₗ^{α} and the selected value of a relative contrast agent enhancement μₜ^{α}. For example, it is possible to calculate, for each of the determined differential values dₗ^{α} a differential value of the metric

m′ₗ^{α}=dₗ^{α}−μₜ^{α}cₗ

wherein the optional constant cₗ can be set to the value one, but it can also show values that differ here from.

If, for example, the subset of the first set of data U₁^{α} and the subset of the second set of data U₂^{α} includes, respectively, k selected data values and if the previously explained k indices iₓ∈{1, . . . , m} are used, it is possible to calculate for each of the k determined differential values dᵢ_{ₓ}^{α} a differential value of the metric

m′ᵢ_{ₓ}^{α}=dᵢ_{ₓ}^{α}−μₜ^{α}cᵢ_{ₓ}.

Based on the differential values of the metric m′ᵢ_{ₓ}^{α} thus calculated, it is possible to determine the metric me^{α}. Thus, the metric can, for example, represent the sum of the amounts of the differential values of the metric or the sum of the squares of the differential values of the metric or any other suitable sum based on the differential values of the metric that represents a measure of the deviation between the differential values dᵢ_{ₓ}^{α} and the selected value of a relative contrast agent enhancement μₜ^{α}.

For example, the metric m^{α} can be calculated as the sum of the squares of the differential values of the metric based on the following exemplary calculation rule:

$${me}^{α} = {\sum }_{x = 1}^{k} {( {m}_{{i}_{x}}^{′ α} )}^{2}$$

However, it is also possible to use other calculation rules for the calculation of m^{α}.

According to an advantageous embodiment, it is suggested that the method include calculating a plurality of metrics, wherein each of the plurality of metrics is associated with a different value of a relative contrast agent enhancement, and wherein the method includes the selection of that value of a relative contrast agent enhancement which is associated with the metric with the smallest deviation measure.

Therefore, it is possible, for example in an iterative method, to keep calculating a metric for a respective value of a relative contrast agent enhancement until the calculated metric corresponds to, for example, a termination condition, wherein the termination condition is selected such that the metric, which represents a measure for the quality of the value of the relative contrast agent enhancement, indicates a sufficient quality of the associated value of a relative contrast agent enhancement.

Thus, a method can, for example, be performed iteratively until a value of a relative contrast agent enhancement μₜ^{α} is selected whose associated metric satisfies the termination condition.

The method can also be performed such that the associated metric is determined, in each case, for a plurality of different values of a relative contrast agent enhancement, wherein the value of the plurality of different values of a relative contrast agent enhancement is selected as finally determined value of a relative contrast agent enhancement, whose metric represents the smallest deviation measure compared to the other metrics of the other values of the relative contrast agent enhancement.

According to an advantageous embodiment, it is suggested that the metric include a filter function, wherein the filter function is set up to frequency-selectively weight data from the first subset of data from the first set of data and data from the subset of data from the second set of data such that at least some data associated with higher frequencies has less influence on the metric than data associated with lower frequencies.

This filter function can be performed, for example, by a corresponding weighting of the individual differential values of the metric m′ᵢ_{ₓ}^{α}by the respective filter coefficient fₓ (with x∈{1, . . . , k}), for example as follows:

m′ᵢ_{ₓ}^{α}=(dᵢ_{ₓ}^{α}−μₜ^{α}cᵢ_{ₓ})fₓ

In doing so, the filter coefficients fₓ are selected, for example, such that a first differential value dᵢ_{ₓ₁}^{α}, which is associated via the index iₓ₁ in the k space with a higher frequency than the frequency associated with a second differential value dᵢ_{ₓ₂}^{α} in the k space (in accordance with the index iₓ₂), is dampened more by the filter coefficient fₓ₁ than the second differential value dᵢ_{ₓ₂}^{α} is dampened by the filter coefficient fₓ₂ associated with said second differential value dᵢ_{ₓ₂}^{α}.

The filter function can thus represent a low-pass filter function. It is thus possible to reduce interferences, such as noise, that occur increasingly in the higher frequency regions of the k space and to thereby improve the determination of the value of the relative contrast agent enhancement for the second time sub-segment.

According to an advantageous embodiment, it is suggested that based on at least one sub-image space in the image space, a sub-image mask associated with the k space be defined in the k space by transforming the at least one sub-image space in the k space, wherein determining a value of a relative contrast agent enhancement associated with the time sub-segment of the second time segment based on data of the subset of data from the first set of data and data of the subset of data from the second set of data and further based on the sub-image space mask associated with the k space.

For example, a sub-image space mask L^{α}=(l₁^{α}, . . . , lₘ^{α}) can be generated in the image space in that this sub-image space mask L_{α}=(l₁^{α}, . . . , lₘ^{α}) in the at least one sub-image space area is set to a predefined value q≠0 while the remaining areas of the image space in the sub-image space mask are set to zero:

$${L}^{α} = ( {l}_{1}^{α} , … , {l}_{m}^{α} ) with {l}_{x} = \{ \begin{matrix}q , & {l}_{x} … selected \\ 0 , & {l}_{x} … non \text{-} selected\end{matrix} ,$$

wherein x∈{1, . . . , ,} applies and lₓ represents the corresponding point in the image space.

Thus, the at least one sub-image space in the image space can be used, for example, to select at least one region of interest, which include, for example, blood vessels, lesions, potential inflammation sites or organs. The smallest possible selection of a sub-image space area is, for example, a single pixel or a single voxel.

The at least one selected sub-image space area, i.e., for example, the sub-image space mask L^{α} which includes this selection information, can then be transformed from the image space into the k space through a corresponding transformation F⁻¹, for example an IFFT or FFT, for the calculation of sub-image space mask M^{α}=(m₁^{α}, . . . , mₘ^{α}) associated with the k space:

M^{α}=F⁻¹L^{α}

Thereafter, this sub-image space mask M^{α} associated with the k space can be used to determine the value of a relative contrast agent enhancement μₜ^{α} associated with the time sub-segment of the second time segment based on data of the subset of data from the first set of data U₁^{α} and on data of the subset of data from the second set of data U₂^{α}.

For this, the determination of the value of a relative contrast agent enhancement μₜ^{α} is performed, for example, in the k space that is at least partially or wholly filtered with the sub-image space mask M^{α} associated with the k space. This means, for example, that portions of the information of the first and of the second set of data, which are associated with an image space area not lying within the at least one selected sub-image space area, do not, or only to a very minor extent, enter into the determination of the value of a relative contrast agent enhancement μₜ^{α}.

Thus, the influence of regions of the MRI data sets of data in which the contrast agent has no or only a very weak effect can be reduced or eliminated for the determination of the value of a relative contrast agent enhancement μₜ^{α} by delimiting these regions by way of the selected at least one sub-image space area and the corresponding sub-image space mask M^{α} associated therewith in the k space, so that the determination of the value of a relative contrast agent enhancement μₜ^{α} can be improved.

For example, the sub-image space mask M^{α} associated with the k space can be used for calculating the metric me^{α} representing a measure for the deviation between the differential values and a value of a relative contrast agent enhancement, so that, for example, the metric me^{α} is calculated at least partially based on the sub-image space mask M^{α} associated with the k space.

If, for example, the subset of the first set of data U₁^{α} and the subset of the second set of data U₂^{α} each contain k selected data values and if the previously explained k indices i_{x∈{}1, . . . , m} (with x∈{1, . . . , k}) are used, the masking data values mᵢ_{₁}^{α}, . . . , mᵢ_{ₖ}^{α} of the sub-image space mask M^{α} associated with the k space, which are associated accordingly in the k space, can be used to calculate the respective differential values, for example, it is possible to calculate, for each of the k determined differential values dᵢ_{ₓ}^{α} a differential value of the metric

m+ᵢ_{ₓ}^{α}=dᵢ_{ₓ}^{α}−μₜ^{α}mᵢ_{ₓ}.

If there is thus a strong filter effect for a point iₓ in the k space as a result of the corresponding masking data value mᵢ_{ₓ}^{α}, the influence of the differential value m′dᵢ_{ₓ}^{α} associated with this point in the k space is reduced.

The metric me^{α} can then, as described above, be calculated based on the differential values m′ᵢ_{ₓ}^{α} which were calculated based on the sub-image space mask M^{α} associated with the k space.

According to an advantageous embodiment, it is suggested that the method include selecting the at least one sub-image space in the image space based on a representation of the first or of the second set of data in the image space.

For example, the first set of data U₁^{α} or the second set of data U₂^{α} can be transformed from the k space into a corresponding set of data in the image space, wherein a suitable transformation can be used for this purpose, such as, for example, an FFT or IFFT. In the following, it will be assumed, without any restrictions, that this is the second set of data U₂^{α}, however, it could also be the first set of data U₁^{α}. FIG. 5 shows an example of such a set of data 510 in the k space, which can be converted into a corresponding second set of data V₂^{α}=(v_{2,1}^{α}, . . . , v_{2,m}^{α}) in the image space by means of a suitable transformation F, such as, for example, by means of an FFT or IFFT:

V₂^{α}=FU₂^{α}

Thereafter, the at least one sub-image space area in the image space is selected based on the transformed set of data V₂^{α}.

This selection can be made, for example, by a user, wherein the transformed set of data V₂^{α} is displayed to the user in the image space, and the user marks the at least one sub-image space area, so that based on this user entry, the corresponding sub-image space mask L^{α} is generated. To select this at least one sub-image space area, it is, however, also possible to use fully or semi-automatic image segmentation algorithms which mark a region as at least one sub-image space area based on the transformed set of data V₂^{α}.

Accordingly, the sub-image space mask L^{α}=(l₁^{α}, . . . , lₘ^{α}) is selected in the image area as follows:

$${L}^{α} = ( {l}_{1}^{α} , … , {l}_{m}^{α} ) with {l}_{x} = \{ \begin{matrix}q , & {v}_{2 , x}^{α} … selected \\ 0 , & {v}_{2 , x}^{α} … non \text{-} selected\end{matrix}$$

Thus, the image information from the first or second set of data can be used to select the at least one sub-image area.

As already explained, the sub-image space mask L^{α} can be transformed from the image space through the transformation F⁻¹ for calculating the sub-image space mask M^{α}=(m₁^{α}, . . . , mₘ^{α}) associated with the k space:

M^{α}=F⁻¹L^{α}

According to an advantageous embodiment, it is suggested that the method comprise determining a value of an absolute contrast agent enhancement based on the determined value of a relative contrast agent enhancement and a lookup database or a mapping function.

This lookup database can, for example, map a non-linear dependence of the values of an absolute contrast agent enhancement on the values of the relative contrast agent enhancement. This non-linear dependence can, for example, be calculated by way of a calibration method using calibration phantoms.

For example, with the help of the lookup database, which may, for example, be a lookup table, it is possible to determine an associated value of an absolute contrast agent enhancement for a value of a relative contrast agent enhancement μₜ^{α}.

For example, it is also possible to use a mapping function f^{L} to determine the value of an absolute contrast agent enhancement based on the determined value of a relative contrast agent enhancement, which mapping function generates, for a value of a relative contrast agent enhancement μₜ^{α} a corresponding associated value of an absolute contrast agent enhancement μ′ₜ^{α} based on a model:

μ′ₜ^{α}=f^{L}(μ′ₜ^{α}),

According to an advantageous embodiment, it is suggested that the method includes determining a plurality of values of a relative contrast agent enhancement for a plurality of time sub-segments of the second time segment based on the data of the subset of data from the first set of data and the data of the subset of data from the second set of data, wherein each of the values of a relative contrast agent enhancement is associated with a different time sub-segment of the plurality of time sub-segments.

For example, it is possible to selected a time sub-segment from the plurality of time sub-segments.

Thereafter, a value of a relative contrast agent enhancement associated with this selected time sub-segment of the second time segment can be determined based on data of a selected subset of data from the first set of data and based on data of a selected subset of data from the second set of data. This determination can be performed by each of the embodiments described above.

For example, it is then possible to verify if there is another time sub-segment of the plurality of time sub-segments, for which a value of a relative contrast agent enhancement needs to be determined.

For example, it is possible to verify if, for a time sub-segment of the plurality of time sub-segments, no value of a relative contrast agent enhancement has been determined yet. If so, a value of a relative contrast agent enhancement associated with this selected time sub-segment of the second time segment is, in turn, determined based on data of a selected subset of data from the first set of data and based on data of a selected subset of data from the second set of data, wherein this determination can be performed by each of the embodiments described above.

If it is determined that no further time sub-segment of the plurality of time sub-segments, for which a relative contrast agent enhancement needs to be determined, the method can, for example, end or, optionally, further process the determined values of a relative contrast agent enhancement.

For example, the second time segment can be subdivided in a plurality of time sub-segments, wherein a corresponding value of a relative contrast agent enhancement is determined for each of the time sub-segments. The plurality of time sub-segments of the second time segment can, for example, also represent a selection of time sub-segments of the second time segment, so that values of relative contrast agent enhancements are determined only for especially selected time sub-segments that do not cover the entire second time segment.

The time sub-segments of the plurality of time sub-segments can, for example, be selected such that at least one time sub-segment of the plurality of time sub-segments is associated, in each case, with exactly one phase step from the plurality of phase steps, and/or that at least one time sub-segment of the plurality of time sub-segments is associated, in each case, with several, preferably adjacent phase steps from the plurality of phase steps.

### BRIEF DESCRIPTION OF THE DRAWINGS

The invention is described hereinafter in detail based on drawings showing embodiments.

The following figures show the following:

FIG. 1a is an example of a system for recording a two-dimensional MRI-scan.

FIG. 1b is an example representing the development of a contrast agent over time after injection of a contrast agent.

FIG. 2 is an example of a method according to a second embodiment.

FIG. 3 is an example of a method according to a third embodiment.

FIG. 4 is an example of a method according to a fourth embodiment.

FIG. 5a is a first example of the calculation of a sub-image space mask according to a fourth embodiment.

FIG. 5b is a second example of the calculation of a sub-image space mask according to the fourth embodiment.

FIG. 6a is an example of a method according to a fifth embodiment.

FIG. 6b is an example of a method according to a sixth embodiment.

FIG. 7 is an example of a method according to a seventh embodiment.

### DETAILED DESCRIPTION OF THE INVENTION

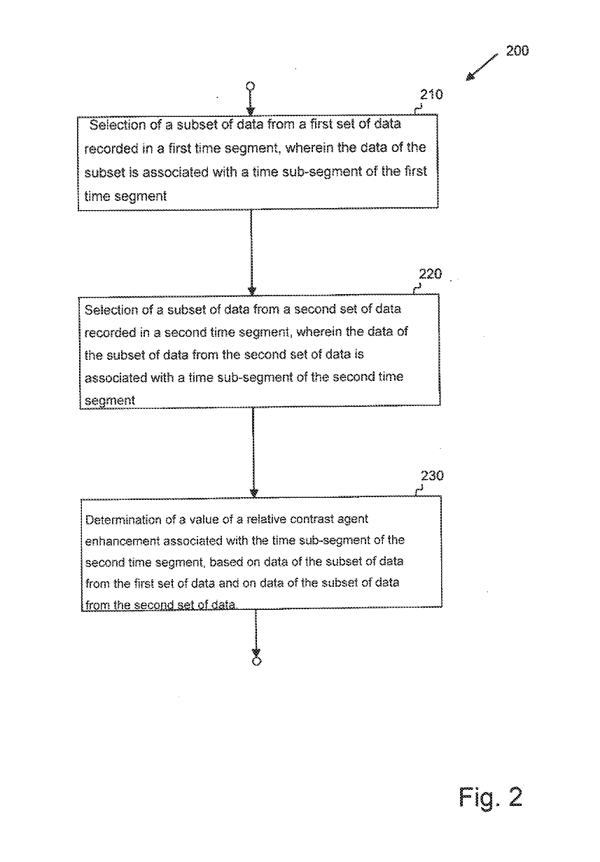

FIG. 2 shows an example of method 200 according to a first embodiment.

Method 200 includes in a step 210 the selecting of a subset of data from a first set of data, wherein the first set of data comprises data of a multi-dimensional k space of a magnetic resonance scan recorded without the influence of a contrast agent, which was recorded in a first time segment.

The multi-dimensional k space can be, for example, a two-dimensional k space corresponding to a two-dimensional MRI scan, wherein the set of data describes a two-dimensional image, or the multi-dimensional k space can also be, for example, a three-dimensional k space corresponding to a three-dimensional MRI scan, wherein the set of data describes a three-dimensional image and thus is able to represent a volume image set of data. The first set of data can be a raw set of data from an MRI scan.

The multi-dimensional k space can, for example, be frequency-encoded with a plurality of frequencies in a first dimension, wherein this plurality of frequencies can represent a plurality of different frequencies with which the MRI scan is recorded.

Furthermore, the multi-dimensional k space can, for example, be encoded in at least on additional direction with a plurality of phase steps. The plurality of phase steps of a respective dimension of the at least one additional dimension can, for example, represent a plurality of different phase values, wherein this plurality of different phase values is associated in each case with different phase encodings in the respective additional dimension, or the plurality of phase steps of a respective dimension of the at least one additional dimension can, for example, also represent a plurality of different phase indices, wherein a phase index of the plurality of different phase indices is, for example, associated with exactly one phase step. Thus it is possible, for example, in the phase encoding in a respective dimension of the at least one additional dimension to run through, for example, all phase indices of the plurality of different phase indices of this respective dimension or through a selection of phase indices of the plurality of different phase indices of these respective dimensions for the respective phase encoding.

Thus, the k space can, for example, represent a two-dimensional k space, wherein a first direction in the k space is frequency-encoded with a plurality of frequencies in accordance with the first dimension, and wherein a second direction in the k space, which is preferably orthogonal to the first direction, is encoded with a plurality of phase steps in accordance with the second dimension.

The k space can, for example, also represent a three-dimensional k space, wherein a first direction in the k space is frequency-encoded with a plurality of frequencies in accordance with the first dimension, and wherein a second direction in the k space, which is preferably orthogonal to the first direction, is encoded with a first plurality of phase steps in accordance with the second dimension, and wherein a third direction in the k space, which is preferably orthogonal to the first and the second direction, is encoded with a second plurality of phase steps in accordance with a third dimension. In this, the phase steps of the plurality of phase steps of the second dimension are distinct, for example, from the phase steps of the third dimension.

Thus, the phase steps of the plurality of phase steps of the second dimension can, for example, be interleaved with the phase steps of the plurality of phase steps of the third dimension. However, it is also possible to use other techniques with regard to the specification of the phase steps of the plurality of phase steps of the second Dimension and the specification of the phase steps of the plurality of phase steps of the third dimension for scanning in the three-dimensional k space.

During the MRI scan, for example, the process runs through the phase encoding steps of the at least one additional dimension in the k space, and while this occurs, a set of data Uₙ^{α} can be recorded in the corresponding k space, wherein the index n stands for the number of the recorded set of data, i.e. for example for the association that the recorded set of data represents the nth set of data, and wherein the optional index a specifies the respective antenna α∈{1, . . . , A} from a number of A antennae, i.e. at least one antenna. Every nth set of data Uₙ^{α} is thus associated with a time segment in which the data of the respective set of data Uₙ^{α} was recorded.

This acquired k space data of the set of data Uₙ^{α} contains, for example, the signal response with regard to amplitude, frequency and phase position during the acquisition.

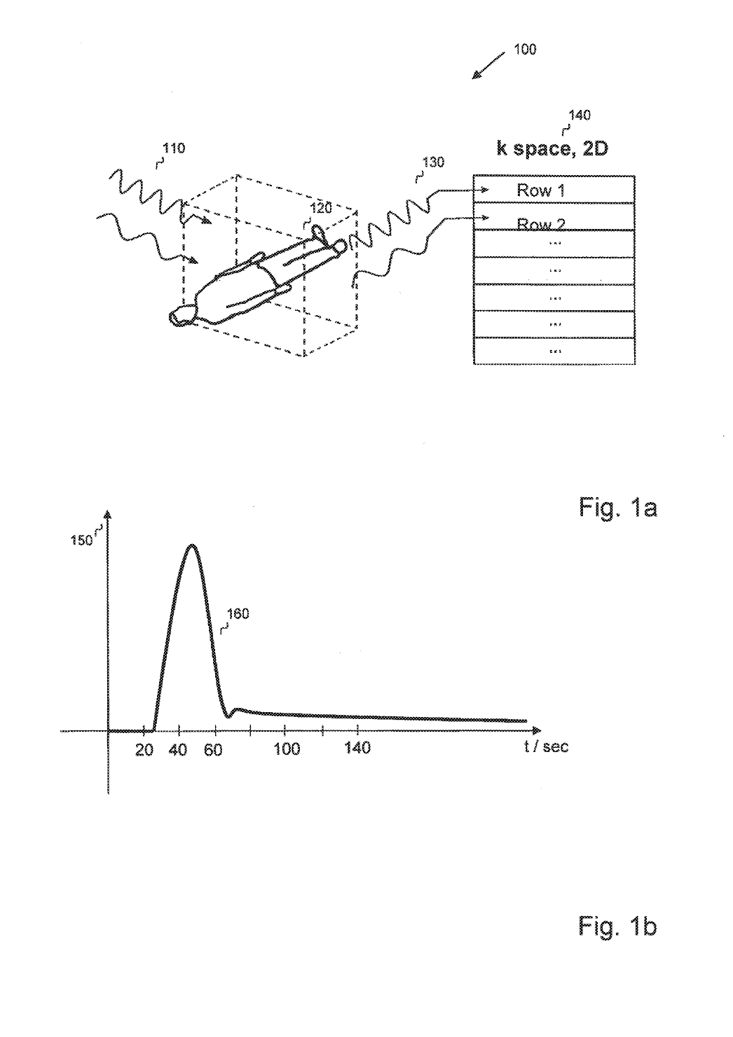

For example, the recording of a set of data Uₙ^{α} in the k space can take place such that the recording starts with a phase step from the plurality of phase steps of one dimension of the at least one additional dimension and for this selected phase step, the data associated accordingly in the k space is recorded, wherein the data recorded for this selected phase step is associated in each case with the various frequencies of the plurality of frequencies, for example via the recording index of the signal received by the receiving antenna. For example, the signal 130 shown in FIG. 1a for the example of the 2D case is associated with a selected phase step, and the data associated with this phase step is recorded in the example in line 1, wherein each recorded data value of this data is associated with, for example, a frequency of the plurality of frequencies, so that then, for example, the different data values of the recorded data are associated each with different frequencies of the plurality of frequencies.

Then it is possible to continue with a further phase step from the plurality of phase steps of one dimension of the at least one additional dimension, wherein, in turn, the data associated accordingly in the k space is recorded for this selected phase step, wherein the data recorded for this selected phase step is, in turn, associated with the various frequencies of the plurality of frequencies. Thus, it is possible to run through each phase step of the plurality of phase steps of each respective dimension of the at least one additional dimension, wherein the data recorded for this selected phase step is associated with different frequencies of the plurality of frequencies, so that, for example, a set of data of a two-dimensional or three-dimensional k space can be recorded. In the two-dimensional k space, the at least one additional dimension comprises, for example, exactly one additional dimension, which is phase-encoded with a plurality of phase steps.

In the three-dimensional k space, the at least one additional dimension comprises, for example, exactly two additional dimensions, wherein a first additional dimension of the two additional dimensions is phase-encoded with a first plurality of phase steps, and a second additional dimension of the two additional dimensions is phase-encoded with a second plurality of phase steps. In this three-dimensional case, a first phase step from the first plurality of phase steps and a second phase step from the second plurality of phase steps is used for phase encoding in the two additional dimensions during an MRI scan, wherein the data then recorded in the k space for this first phase step of the first additional dimension and for this phase step of the second additional dimension, are in each case associated in turn with the various frequencies of the plurality of frequencies. The example of a 2D recording shown in FIG. 1a can therefore also be transferred to a 3D recording, wherein the received signal 130 is associated with a first phase step from the first plurality of phase steps and simultaneously with a second phase step from the second plurality of phase steps, and wherein, for example, the data associated with this phase step is recorded in the example in line 1, wherein each recorded data value of this data is associated with, for example, a frequency of the plurality of frequencies, so that then, for example, the different data values of the recorded data are associated each with different frequencies of the plurality of frequencies. Thus a complete 3D scan can be generated for example by combining all phase steps of the first plurality of phase steps with each respective phase step of the second plurality of phase steps into a pair of phase steps, and by recording the data for each combination in the k space, which data in turn is associated with the various frequencies of the plurality of frequencies.

The data of the acquired set of data Uₙ^{α} is therefore associated with a time segment in which the recording of the set of data Uₙ^{α} has occurred while running through the individual phase steps or combinations of pairs of phase steps of the first and second additional dimension in the k space, wherein, for example, the data recorded, in each case, for one phase step or for a pair of phase steps and associated with the respective different frequencies can be associated with the approximately same point in time or different points in time. For example, a set of data Uₙ^{α} can comprise a total of m successive data values in the k space, which are, for example, recorded during the MRI recording by sampling:

Uₙ^{α}=(u_{n,1}^{α}, . . . , u_{n,m}^{α})

For example, in the 2D case, a respective data value u_{n,l}^{α} (with l∈{1, . . . , m}) of the plurality of data values Uₙ^{α}=(u_{n,1}^{α}, . . . , u_{n,m}^{α}) can be associated with exactly one phase step and exactly one frequency, or in the 3D case, a respective data value u_{n,l}^{α} (with l∈{1, . . . , m}) of the plurality of data values Uₙ^{α}=(u_{n,1}^{α}, . . . , u_{n,m}^{α}) can be associated, for example, with exactly one phase step from the first plurality of phase steps, exactly one second phase step from the second plurality of phase steps, i .e . a pair of phase steps of the first and second additional dimension, and exactly one frequency. Furthermore, a respective data value u_{n,l}^{α} of the plurality of data values Uₙ^{α}=(u_{n,1}^{α}, . . . , u_{n,m}^{α}) can, for example, be associated with exactly one recording time within the time segment of the set of data with l∈{1, . . . , m}.

Depending on the MRI method used, it is also possible, for example, for several different data values u_{n,l}^{α} of the plurality of data values Uₙ^{α}=(u_{n,1}^{α}, . . . , u_{n,m}^{α}) to be associated with the same point in time, or substantially the same point in time. For example, data values u_{n,l}^{α}, which are associated with the same phase step or the same pair of phase steps and, in each case, different frequencies, can be associated with the same or substantially the same point in time, while data values u_{n,l}^{α}, which are associated with different phase steps or different pairs of phase steps, can be associated, in each case, with different points in time, in accordance with the temporal occurrence of the respective phase step or the respective pair of phase steps.

However, it is also possible that all data values u_{n,l}^{α} of a set of data Uₙ^{α} are each associated with different points in time within the time segment, wherein the time interval between the recorded data values may be the same, or wherein the time interval may, depending on the MRI method used, however also vary between different adjacent data values. Each of the data values is associated with one point in time within the time segment of the set of data Uₙ^{α}.

The representation of the data values of the set of data Uₙ^{α} may also deviate from (3), for example by a two or multi-dimensional matrix, in which the data values are deposited for example in columns and rows, i.e. a respective data value u_{n,l}^{α} (with l∈{1, . . . , m}) of the plurality of data values Uₙ^{α}=(u_{n,l}^{α}, . . . , u_{n,m}^{α}) can, for example, be associated with a column and a row. The plurality of data values Uₙ^{α}=(u_{n,1}^{α}, . . . , u_{n,m}^{α}) can therefore also be considered a special representation of a multi-dimensional k space, such as a two or three-dimensional k space.

Thus this time segment can be, for example in a three-dimensional gradient echo sequence for an MRI mammography, between about 40 s and 120 s long, wherein the time segment may also have values deviating here from.

The first set of data used step 210, which was recorded without the influence of a contrast agent and which is associated with a first time segment, can, for example, be represented as U₁^{α}.

In step 210, a first subset of data from the first set of data U₁^{α} is selected, wherein the data of the subset is associated with a time sub-segment of the first time segment. The subset of data thus contains fewer data values than the total first set of data U₁^{α}, i.e. the time sub-segment represents an excerpt of the first time segment. For example, the subset of data comprise those data values u_{1,t}^{α} with l∈{1, . . . , m}, whose time points lie within the time sub-segment. For example, all data values from the first set of data U₁^{α} which lie within the time sub-segment can be selected as subset of data, or a selection of data values from the totality of the data values of the first set of data which lie within the time sub-segment can be selected as subset of data.

Thus, the subset of data can, for example, comprise a total of k selected data values of the first set of data U₁^{α} with k<m, whose time points lie within the time sub-segment, wherein, for example, the selected data values can be specified through a quantity of k indices iₓ∈{1, . . . , m} with x∈{1, . . . , k} so that for example a respective xth index iₓ is clearly associated with one of the data values from the total of k selected data values of the first set of data U₁^{α}, so that the data value u_{1,i}_{ₓ}^{α} corresponds to this data value. Therefore, the data values u_{1,i}_{₁}^{α}, . . . , u_{1,i}_{ₖ}^{α} represent the k selected data values of the first set of data U₁^{α}, wherein the selection of the data values of the first set of data U₁^{α} can be made by associating the k indices iₓ with the corresponding indices {1, . . . , m} in the k space.

Method 200 further includes in a step 220 the selecting of a subset of data from a second set of data, wherein the second set of data comprises data of a multi-dimensional k space of a magnetic resonance scan recorded with the influence of a contrast agent, which was recorded in a second time segment. This second set of data can, for example, be denoted as U₂^{α}, wherein the MRI scan can be recorded in the k space in analogy to the general procedure described above.

This second time segment can, for example, be situated in time after the first time segment. Thus this first set of data can be, for example, the MRI scan of a patient without the use of a contrast agent, wherein the patient is injected with a contrast agent following this MRI scan, and after the injection of the contrast agent, a second MRI scan is done of the same patient, preferably in the same position as during the first MRI scan, for the recording of the second set of data.

However, the second set of data can, for example, also be recorded prior to the first set of data, i.e. the first time segment can, for example, also be situated in time after the second time segment. Thus, a patient can, for example, be injected with a contrast agent first, and after the injection of the contrast agent, an MRI scan of the patient is performed to record the second set of data. Then one waits until the injected contrast agent no longer has any significant effect on an MRI scan, so that then another MRI scan of the same patient is performed, if possible in the same position, to record the first set of data. Therefore, the recording of the first set of data without the influence of a contrast agent can, for example, also be understood such that then the effect of the contrast agent is very low compared to the freshly injected contrast agent.

The subset of data from the second set of data U₂^{α} thus contains fewer data values than the entire second set of data U₂^{α}, i.e. the time sub-segment represents an excerpt of the second time segment. For example, the subset of data can comprise those data values u_{n,l}^{α} with l∈{1, . . . , m} whose time points lie within the time sub-segment of the second time segment. For example, all data values from the second set of data U₂^{α} which lie within the time sub-segment of the second time segment can be selected as subset of data, or a selection of data values from the totality of the data values of the second set of data which lie within the time sub-segment of the second time segment can be selected as subset of data.

The relative temporal position of the time sub-segment of the second time segment can therefore correspond to the relative temporal position of the time sub-segment of the first time segment.

The data of the subset of data from the first set of data U₁^{α} selected in step 210, and the data of the subset of data from the second set of data U₂^{α} selected in step 220 are selected such that the data of the subset of data from the first set of data U₁^{α} is associated with substantially the same region in the k space as the data of the subset of data from the second set of data U₂^{α}.

If, for example, a data value of the subset of data from the second set of data U₂^{α} selected in step 220 represents the data value u_{2,l}^{α} with l∈{1, . . . , m}, then, for example, the data value of the subset of data from the first set of data located in the same position in the k space can be selected by the corresponding data value u_{1,l}^{α}, since the same index l represents the same point in the k space. Therefore, for example, the subset of data of the first set of data can have, for each data value u_{2,l}^{α} of the subset of data selected in step 220, a data value u_{1,l}^{α} located, in each case, at the same, or substantially the same, point in the k space.

If the subset of data of the first set of data comprises, for example, a total of k selected data values of the first set of data U₁^{α} with k<m, with the data values u_{1,i}_{₁}^{α}, . . . , u_{1,i}_{ₖ}^{α} representing the k selected data values of the first set of data U₁^{α}, the selected data values of the second set of data U₂^{α} can, for example, can be specified through the set of k indices iₓ∈{1, . . . , m} with x∈{1, . . . , k} already used for the subset of data of the first set of data, so that u_{2,i}_{₁}^{α}, . . . , u_{2,i}_{ₖ}^{α} represent the k selected data values of the second set of data U₂^{α}.

The data of the subset of data from the first set of data U₁^{α} can comprise several data values and the data of the subset of data from the second set of data U₂^{α} can comprise several data values. Thus, k≧2 may apply, and/or, if for example all data values from the first set of data U₁^{α} which lie in the time sub-segment represent the subset of data from the first set of data U₁^{α}, and if, for example, all data values from the second set of data U₂^{α} which lie in the time sub-segment of the second time segment represent the subset of data from the second set of data U₂^{α}, m≧2 may apply.

The order of steps 210 and 220 is interchangeable. Therefore, it is also possible to first select the subset of data of the second set of data (step 220) and then, for example, the subset of data of the first set of data selected (step 210). If the data values u_{2,i}_{₁}^{α}, . . . , u_{2,i}_{ₖ}^{α} represent the k data values of the subset of the second set of data U₂^{α}, for example selected in step 220, the data values of the subset of the first set of data U₁^{α} can, for example, be specified through the set of k indices iₓ∈{1, . . . , m} with x∈{1, . . . , k} already used for the subset of data of the first set of data, so that u_{1,i}_{₁}^{α}, . . . , u_{1,i}_{ₖ}^{α} represent the k data values of the first set of data U₁^{α}, which are selected in step 210.

It is, for example, also possible for the selection of the subset of data of the second set of data and the selection of the subset of data of the first set of data in steps 220 and 210 to occur simultaneously, wherein for a selected data value of the subset of the first or second set of data, the corresponding associated data value of the subset of the second or first set of data is selected directly.

Then the method comprises in a step 230 the determining of a value of a relative contrast agent enhancement associated with the time sub-segment of the second time segment which is calculated based on data of the subset of data from the first set of data U₁^{α} and on data of the subset of data from the second set of data U₂^{α}. Thus, for example, the several data values of the first subset of data from the first set of data U₁^{α} and the several data values of the second subset of data from the second set of data U₂^{α} can be used to determine the value of a relative contrast agent enhancement associated with the time sub-segment of the second time segment.

In this, a single value of a relative contrast agent enhancement is, for example, calculated for the time sub-segment of the second time segment, hereinafter always referred to as second time sub-segment, wherein the information is exploited that the MRI scan of the first set of data was done without contrast agent and the MRI scan of the second set of data under the influence of the contrast agent.

As in determining the value of a relative contrast agent enhancement from the second set of data the data used is exclusively that which was recorded during the second time sub-segment—i.e. data from the second set of data lying within the second time segment, but outside of the second time sub-segment, is not used—, the contrast agent enhancement present in the patient's body during this second time sub-segment is the primary factor influencing the determination of the value of a relative contrast agent enhancement.

Therefore, the present method allows for the temporal resolution of the value of a relative contrast agent enhancement determined in step 230 to be improved compared to traditional methods which determine the value of a relative contrast agent enhancement based on all data acquired in the k space.

The determination of the value of a relative contrast agent enhancement associated with the second time sub-segment made in step 230 can be performed through various methods.

Thus, for example, the differential values between data of the subset of the second set of data U₂^{α} and data of the subset of the first set of data U₁^{α} can be formed, wherein a respective differential value d₁^{α} based on the difference of a data value u_{2,l}^{α} of the data of the subset of the second set of data U₂^{α} and of the data value u_{1,i}^{α} of the data of the subset of the first set of data U₁^{α} associated with said data value u_{2,l}^{α} in the k space, for example by

dₗ^{α}=u_{2,l}^{α}−u_{1,l}^{α}.

If, for example, the subset of the first set of data U₁^{α} and the subset of the second set of data U₂^{α} includes respective k selected data values and if the previously explained k indices iₓ∈{1, . . . , m} are used, an xth differential value (with x∈{1, . . . , k}) of the in total k differential values can be calculated as follows:

dᵢ_{ₓ}^{α}=u_{2,i}_{ₓ}^{α}−u_{1,i}_{ₓ}^{α}.

The respective data value u_{1,i}_{ₓ}^{α} of the data of the subset of the first set of data U₁^{α} contains, for example, the signal response in terms of amplitude, frequency and phase position during the acquisition in the k space associated with the data value u_{2,i}_{ₓ}^{α}, wherein the signal response does not comprise any portion caused by a contrast agent. In comparison to the respective data value u_{1,i}_{ₓ}^{α} of the data of the subset of the first set of data U₁^{α}, the associated differential value u_{2,i}_{ₓ}^{α} of the data of the subset of the second set of data U₂^{α} contains an additional signal component Δ₁^{α}, which is proportional to the relative contrast agent enhancement or represents a value of a relative contrast agent enhancement:

u_{2,i}_{ₓ}^{α}=u_{1,i}_{ₓ}^{α}+Δᵢ_{ₓ}^{α}

This additional signal component Δᵢ_{ₓ}^{α} can be determined, for example, by forming the differential value dᵢ_{ₓ}^{α}, and can be seen, for example, as an estimation of the relative contrast agent enhancement for the point in the k space associated with the differential value dᵢ_{ₓ}^{α}.

Each of the determined differential values dᵢ_{ₓ}^{α} can therefore be considered an estimation of the relative contrast agent enhancement in the respective point in the k space, so that it is possible to determine, for example based on an averaging of the determined differential values (or of the amounts of the determined differential values), the value of a relative contrast agent enhancement associated with the second time sub-segment. If only a single differential value is determined, this differential value or the amount of this differential value can be seen, for example, as the value of a relative contrast agent enhancement associated with the second time sub-segment.

However, it is also possible to use other methods for determining the value of a relative contrast agent enhancement associated with the second time sub-segment.

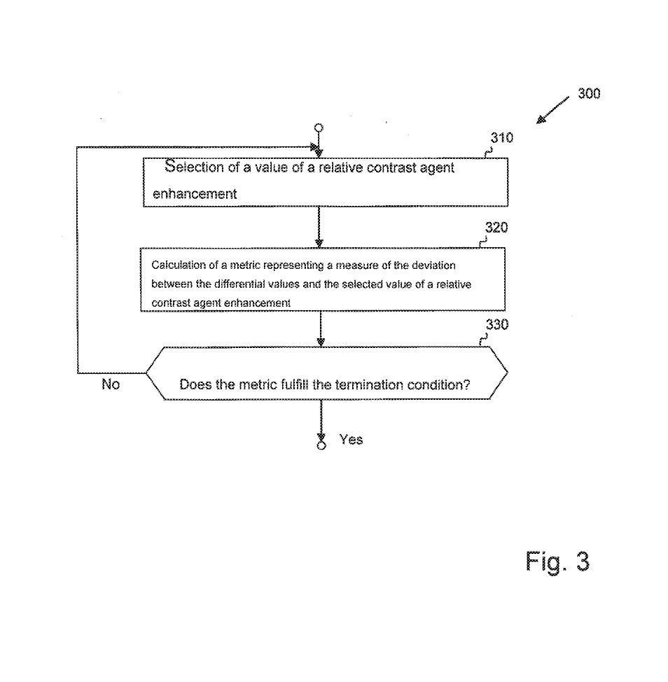

FIG. 3 shows an example of method 300 according to a third embodiment.

This method 300 can be used, for example, to determine a value of a relative contrast agent enhancement associated with the time sub-segment of the second time segment based on data of the subset of data from the first set of data and data of the subset of data from the second set of data in step 230 of method 200 according to the first embodiment.

To determine the value of a relative contrast agent enhancement associated with the second time sub-segment, method 300 uses in step 230 the calculation of a metric based on the determined differential values, wherein the metric represents a measure for the deviation between the differential values and a value of a relative contrast agent enhancement. These differential values represent, for example, the previously described differential values between data of the subset of the second set of data U₂^{α} and data of the subset of the first set of data U₁^{α}.

In step 310, a value of a relative contrast agent enhancement is selected. Thus, a suitable starting value for the estimation of the value of a relative contrast agent enhancement can, for example, be used here at the beginning of method 300.

Then, the metric for this selected value of a relative contrast agent enhancement is determined in step 320, which metric represents a measure for the deviation between the differential values and the selected value of a relative contrast agent enhancement, in the following always referred to as μₜ^{α}. The index t of the value of a relative contrast agent enhancement μₜ^{α} is an indicator for the time sub-segment of the second time segment.

For example, the metric can be calculated by calculating a differential value of the metric m′ₗ^{α} for each of the determined differential values dₗ^{α}, which is calculated based on the difference between the respective differential value dₗ^{α} and the selected value of a relative contrast agent enhancement μₜ^{α}. For example, it is possible to calculate, for each of the determined differential values dₗ^{α} a differential value of the metric

m′ₗ^{α}=dₗ^{α}−μₜ^{α}cₗ

wherein the optional constant cₗ can be set to the value one, but it can also show values that differ here from.

If, for example, the subset of the first set of data U₁^{α} and the subset of the second set of data U₂^{α} includes, respectively, k selected data values and if the previously explained k indices iₓ∈{1, . . . , m} are used, it is possible to calculate for each of the k determined differential values dᵢ_{ₓ}^{α}. a differential value of the metric

m′ᵢ_{ₓ}^{α}=dᵢ_{ₓ}^{α}−μₜ^{α}cᵢ_{ₓ}.

Based on the differential values of the metric m′ᵢ_{ₓ}^{α} thus calculated, it is possible to determine the metric me^{α} in step 320. Thus, the metric can, for example, represent the sum of the amounts of the differential values of the metric or the sum of the squares of the differential values of the metric or any other suitable sum based on the differential values of the metric that represents a measure of the deviation between the differential values dᵢ_{ₓ}^{α} and the selected value of a relative contrast agent enhancement μₜ^{α}.

For example, the metric m^{α} can be calculated as the sum of the squares of the differential values of the metric based on the following calculation rule:

$${me}^{α} = {\sum }_{x = 1}^{k} {( {m}_{{i}_{x}}^{′ α} )}^{2}$$

In step 330 it is then possible to verify, for example, whether the metric me^{α} calculated in step 320 satisfies a termination condition, i.e. whether the measure of deviation calculated by the metric me^{α} lies below a set limit value.

If the termination condition is not fulfilled, i.e. if the metric represents a too poor measure for the deviation between the differential values and the value of a relative contrast agent enhancement μₜ^{α} selected in step 310, the value of a relative contrast agent enhancement μₜ^{α} selected in step 310 is not sufficient as finally determined value of a relative contrast agent enhancement, and the method starts again at step 310 with the selection of a new, other value of a relative contrast agent enhancement.

Thus, method 300 can, for example, be performed iteratively until in step 310, a value of a relative contrast agent enhancement μₜ^{α} is selected whose associated metric, calculated in step 320, satisfies the termination condition in step 330.

The method shown in FIG. 3 can also be modified such that the associated metric is determined, in each case, for a plurality of different values of a relative contrast agent enhancement, in analogy to the calculation in step 320, wherein the value of the plurality of different values of a relative contrast agent enhancement is selected as finally determined value of a relative contrast agent enhancement, whose metric represents the smallest deviation measure compared to the other metrics of the other values of the relative contrast agent enhancement.

For example, the metric calculated in step 320 can also include an optional filter function, wherein the filter function is set up to frequency-selectively weight data values from the first subset of data from the first set of data and data from the subset of data from the second set of data, wherein said frequency-selective weighting can be achieved, for example, through a band pass filter function, such that, for example, at least some data associated with high frequencies, and at least some data associated with low frequencies, has less influence on the metric than data associated with frequencies between the high and the low frequencies.

This filter function can be performed, for example, by a corresponding weighting of the individual differential values of the metric m′ᵢ_{ₓ}^{α} by the respective filter coefficient fₓ (with x∈{1, . . . , k}), for example as follows:

m′ᵢ_{ₓ}^{α}=(dᵢ_{ₓ}^{α}−μₜ^{α}cᵢ_{ₓ})fₓ

In doing so, the filter coefficients fₓ are selected, for example, such that a first differential value dᵢ_{ₓ₁}^{α}, which is associated via the index iₓ₁ in the k space with a higher frequency than the frequency associated with a second differential value dᵢ_{ₓ₂}^{α} in the k space (in accordance with the index iₓ₂), is dampened more by the filter coefficient fₓ₁ than the second differential value dᵢ_{ₓ₂}^{α} is dampened by the filter coefficient fₓ₂ associated with said second differential value dᵢ_{ₓ₂}^{α}, and that a third differential value dᵢ_{ₓ₃}^{α}, which is associated via the index iₓ₃, in the k space with a lower frequency than the frequency associated with the second differential value dᵢ_{ₓ₂}^{α} in the k space (in accordance with the index iₓ₂), is dampened more by the filter coefficient fₓ₃ than the second differential value dᵢ_{ₓ₂}^{α} is dampened by the filter coefficient fₓ₂ associated with said second differential value dᵢ_{ₓ₂}^{α}.

The filter function can thus represent a band-pass filter function. It is thus possible to reduce interferences, such as noise, that occur increasingly in the higher frequency regions and to thereby improve the determination of the value of the relative contrast agent enhancement performed in step 230 for the second time sub-segment. Alternatively, it is also possible to use, for example, a low-pass filter function.

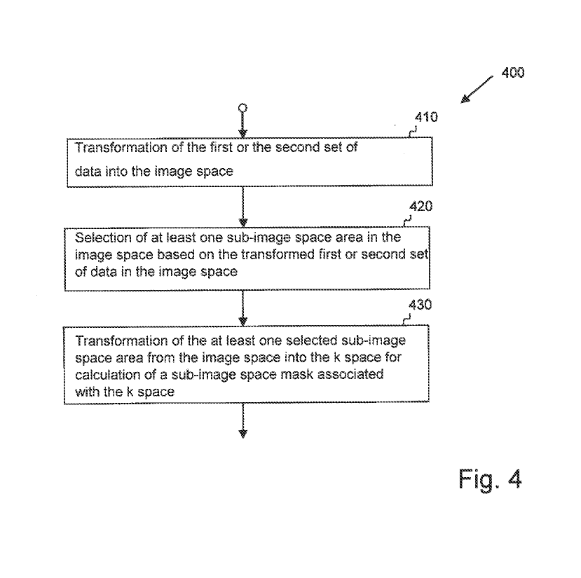

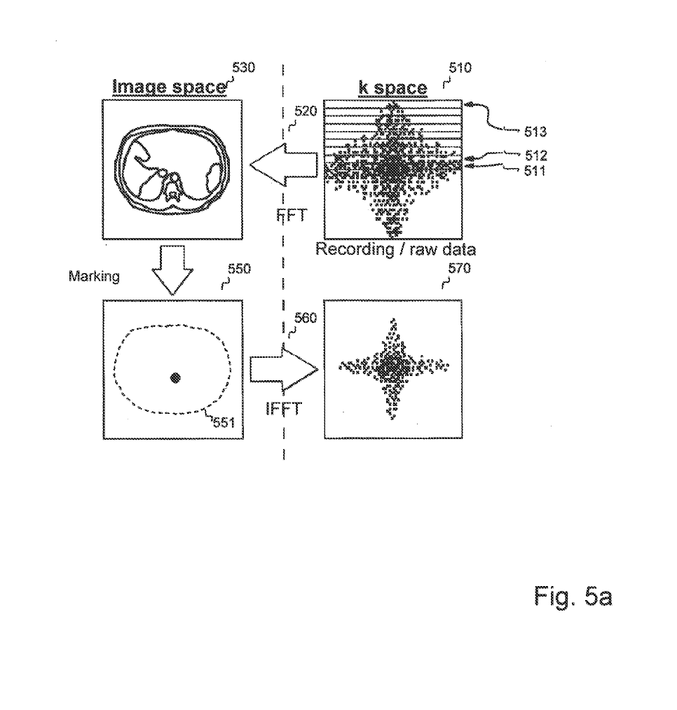

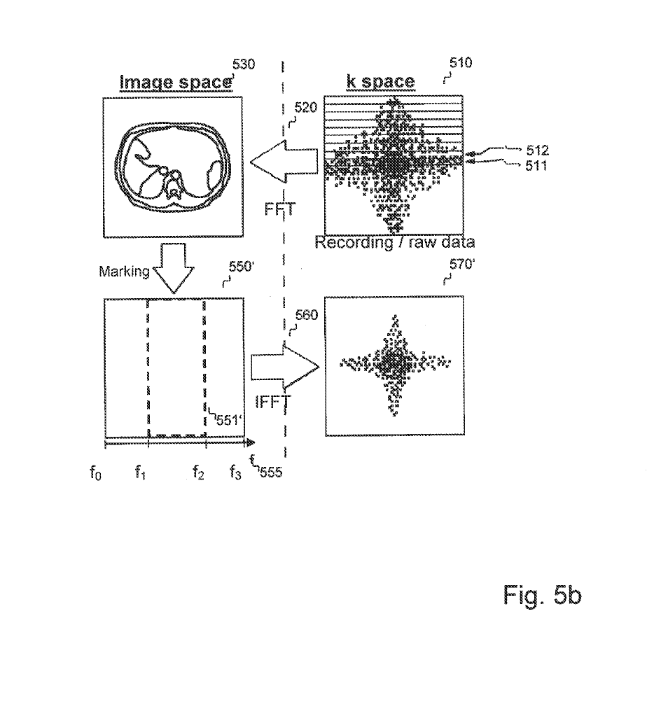

FIG. 4 shows an example of a method 400 according to a fourth embodiment. This method 400 is explained together with the first example, shown in FIG. 5a, of a calculation of a sub-image space mask according to a fourth embodiment and the second example, shown in FIG. 5b, of a calculation of a sub-image space mask according to a fourth embodiment.

In step 410, the first set of data U₁^{α} or the second set of data U₂^{α} is transformed from the k space into a corresponding set of data in the image space. In the following, it will be assumed, without any restrictions, that this is the second set of data U₂^{α}, however, it could also be the first set of data U₁^{α}. FIG. 5a or FIG. 5b. show an example of such a set of data 510 in the k space. This set of data can, for example, be formed from a plurality of rows, wherein each row is associated with a phase step or a pair of phase steps, wherein the data values of a row are associated with the various frequencies from the plurality of the frequencies of the k space. In the example of the 2D case, row 511 can, for example, be associated with the first phase step from the plurality of the phase steps, row 512 can, for example, be associated with the second phase step of the plurality of the phase steps, and so on.

The set of data 510 can be converted into a corresponding set of data V₂^{α}=(v_{2,1}^{α}, . . . , v_{2,m}^{α}) in the image space by means of a suitable transformation F 520, such as, for example, an FFT or IFFT:

V₂^{α}=FU₂^{α}

In step 420, at least one sub-image space area is selected in the image space based on the transformed set of data V₂^{α}.

For example, this selecting of at least one sub-image space in the image space can be done by setting a sub-image space mask L^{α}=(l₁^{α}, . . . , lₘ^{α}) in the at least one sub-image space area to a predefined value q≠0 while the remaining areas of the image space in the sub-image space mask are set to zero:

$${L}^{α} = ( {l}_{1}^{α} , … , {l}_{m}^{α} ) with {l}_{x} = \{ \begin{matrix}q , & {v}_{2 , x}^{α} … selected \\ 0 , & {v}_{2 , x}^{α} … non \text{-} selected\end{matrix}$$

The predefined value q can, for example, be set to one, or to another constant value that is not zero.

This selecting of the at least one sub-image space in the image space can be used, for example, to select at least one region of interest, which include, for example, blood vessels, lesions, potential inflammation sites or organs. The smallest possible selection of a sub-image space area is, for example, a single pixel or a single voxel.

FIG. 5a or FIG. 5b show an example of a set of data V₂^{α} transformed to the image space (see reference number 530), wherein in FIG. 5a, a sub-image space area 551 is selected in the image space 550 based on this transformed set of data V₂^{α}, and in FIG. 5b, a sub-image space area 551′ is selected in the image space 550 based on this transformed set of data V₂^{α} to generate a sub-image space mask, as previously described by way of an example.

This selection can be made, for example, by a user, wherein the transformed set of data V₂^{α} is displayed to the user in the image space, and the user marks the at least one sub-image space area 551, so that based on this user entry, the corresponding sub-image space mask L^{α} is generated. To select this at least one sub-image space area 551, it is, however, also possible to use fully or semi-automatic image segmentation algorithms which mark a region as at least one sub-image space area 551 based on the transformed set of data V₂^{α}.

Mathematically, the k space can, for example, be seen as being associated with a time domain, because the temporally received signal is being scanned here and therefore, data values associated with certain points in time are being recorded, as already explained in detail, even if these data values are each associated with a phase step or a pair of phase values and a frequency in the k space. In mathematical terms, a transformation from the time domain into a frequency domain is performed by the transformation 520, wherein this transformation is performed, for example, for each of the rows represented in the k space (such as, for example 511, 512). A row in the image space 530 transformed accordingly can therefore be associated with a real frequency spectrum, which is not related to the frequencies of the k space. Therefore, the image space 530 can, for example as shown in the example in FIG. 5b, be associated with a frequency spectrum 555, wherein data values of the image space are associated with a frequency from this frequency spectrum.

Therefore, is possible, for example in accordance with one embodiment, to choose the at least one sub-image space areas in the image space in step 420 such that the at least one sub-image space area is not associated with at least one frequency sub-spectrum of the frequency spectrum of the image space, i.e. that the frequencies of the at least one sub-image space areas from the frequency spectrum are not associated with the at least one frequency sub-spectrum. As shown in the image space 550′ in the example in FIG. 5b, one first frequency sub-spectrum could, for example, lie between f₀ and f₁ of the frequency spectrum which, in the example, lies between f₀ and f₃, and a second frequency sub-spectrum can lie between f₂ and f₃, wherein a sub-image space area 551′ is selected such that it does not lie within the first frequency sub-spectrum and the second frequency sub-spectrum.

For example, a frequency sub-spectrum can be selected such that the frequency of an interferer irradiating form the outside and interfering with the MRI scan lies within this frequency sub-spectrum, so that this interference can be hidden for the subsequent determination of the relative value of a contrast agent enhancement.

For example, it is thus possible to select a region of interest in the image space 550′ as sub-image space mask, wherein any points in the image space 550′ that lie in one of the at least one frequency sub-spectrums are not selected in the sub-image space mask 550′.

In step 430, the at least one selected sub-image space area, i.e., for example, the sub-image space mask which includes this selection information L^{α}, is transformed from the image space into the k space through a corresponding transformation 560, for example an IFFT or FFT, which represents an inverse transformation F⁻¹ to transformation 520, for the calculation of sub-image space mask M^{α}=(m₁^{α}, . . . , mₘ^{α}) associated with the k space:

M_{α}=F⁻¹L^{α}

Thereafter, this sub-image space mask M^{α} associated with the k space, represented in FIG. 5, as an example, by reference number 570, can be used, for example in step 230 of the method 200 shown in FIG. 2, to determine the value of a relative contrast agent enhancement μₜ^{α} associated with the time sub-segment of the second time segment is calculated based on data of the subset of data from the first set of data U₂^{α} and on data of the subset of data from the second set of data U₂^{α}.

For this, the determination of the value of a relative contrast agent enhancement μₜ^{α} is performed, for example, in the k space that is at least partially or wholly filtered with the sub-image space mask M^{α} associated with the k space. This means, for example, that portions of the information of the first and of the second set of data, which are associated with an image space area not lying within the at least one selected sub-image space area, do not, or only to a very minor extent, enter into the determination of the value of a relative contrast agent enhancement μₜ^{α}.

Thus, the influence of regions of the MRI set of datas in which the contrast agent has no or only a very weak effect can be reduced or eliminated for the determination of the value of a relative contrast agent enhancement μₜ^{α} by delimiting these regions by way of the selected at least one sub-image space area and the corresponding sub-image space mask M^{α} associated therewith in the k space, so that the determination of the value of a relative contrast agent enhancement μₜ^{α} can be improved.

For example, the sub-image space mask M^{α} associated with the k space can be used for calculating the metric me^{α} representing a measure for the deviation between the differential values and a value of a relative contrast agent enhancement, so that, for example, the metric me^{α} calculated in step 320 of method 300 is calculated at least partially based on the sub-image space mask M^{α} associated with the k space.

If, for example, the subset of the first set of data U₁^{α} and the subset of the second set of data U₂^{α} each contain k selected data values and if the previously explained k indices iₓ∈{1, . . . , m} (with x ∈{1,. . . , k}) are used, the masking data values mᵢ_{₁}^{α}, . . . , mᵢ_{ₖ}^{α} of the sub-image space mask M^{α} associated with the k space, which are associated accordingly in the k space, can be used to calculate the respective differential values, for example, it is possible to calculate, for each of the k determined differential values dᵢ_{ₓ}^{α} a differential value of the metric

M′ᵢ_{ₓ}^{α}=dᵢ_{ₓ}^{α}−μₜ^{α}mᵢ_{ₓ}.

If there is thus a strong filter effect for a point iₓ in the k space as a result of the corresponding masking data value mᵢ_{ₓ}^{α}, the influence of the differential value m′dᵢ_{ₓ}^{α} associated with this point in the k space is reduced.

The metric me^{α} can then, as described above, be calculated based on the differential values m′ᵢ_{ₓ}^{α} which were calculated based on the sub-image space mask M^{α} associated with the k space.

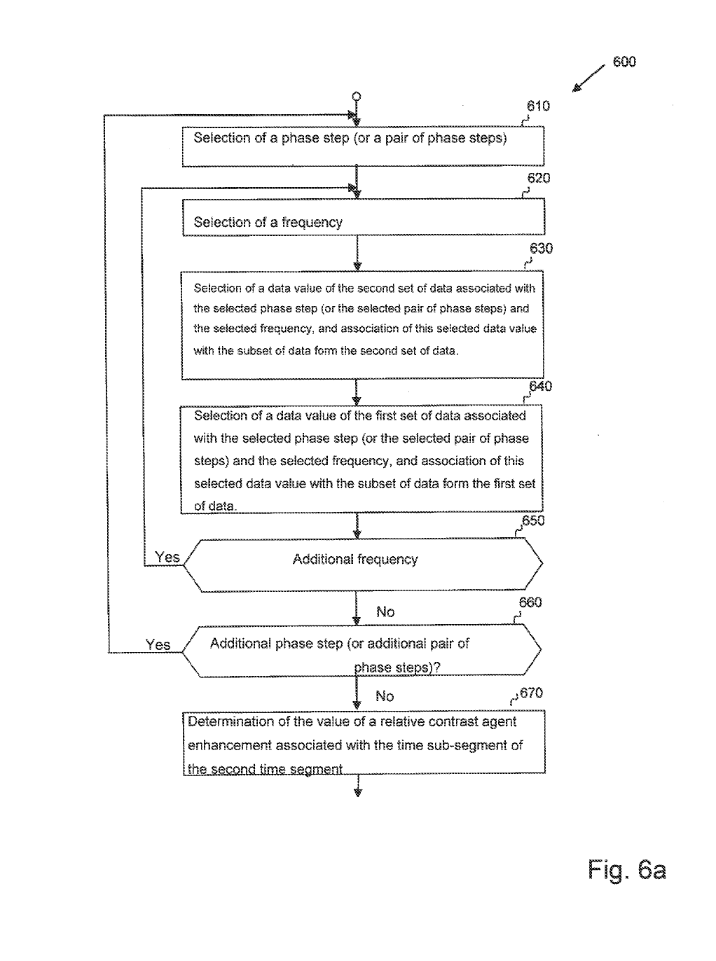

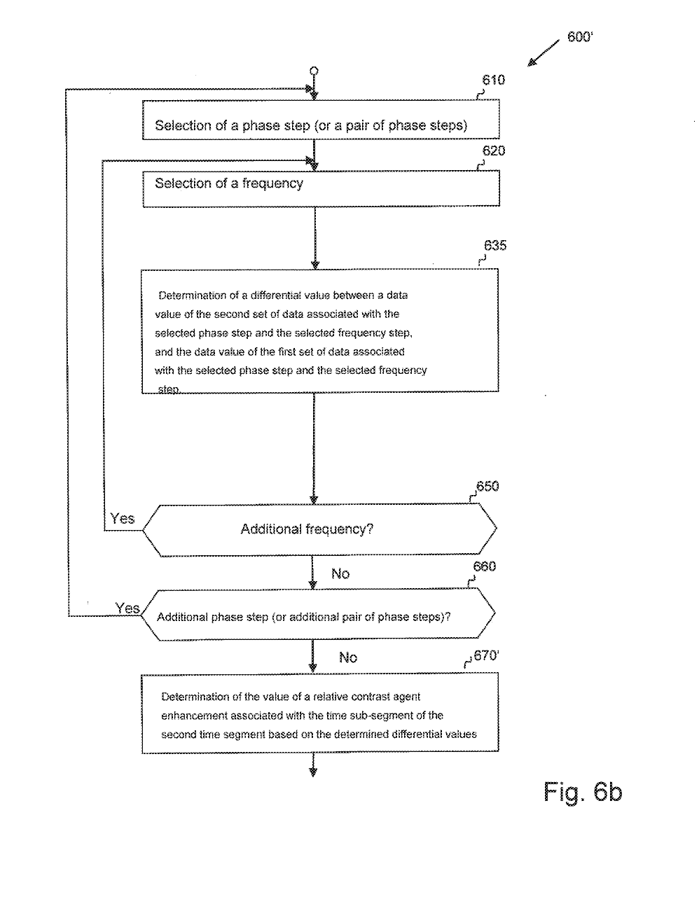

FIG. 6a shows an example of a method 600 according to a fifth embodiment;

This method 600 can be used, for example, for selecting a subset of data from the first set of data according to step 210 from the method 200 shown in FIG. 2 and for selecting a subset of data from the second set of data according to step 220 and for determining a value of a relative contrast agent enhancement associated with the time sub-segment of the second time segment according to step 230.

The exemplary method 600 is based, for example, on the assumption that the recording of a set of data Uₙ^{α} in the k space can take place such that the recording starts with a phase step from the plurality of phase steps of one dimension of the at least one additional dimension, wherein, for example in the 3D case, this phase step can be a phase step of a pair of phase steps of the first additional and the second additional dimension, and that the data in the k space associated with the individual frequencies of the plurality of frequencies is recorded for this selected phase step (or in the 3D case, pair of phase steps). Then it is possible to continue with a further phase step from the plurality of phase steps of one dimension of the at least one additional dimension, wherein, in the 3D case, this further phase step can be a phase step of a further pair of phase steps of the first additional and the second additional dimension, wherein, in turn, the data in the k space associated with the individual frequencies of the plurality of frequencies is recorded for this selected phase step (or in the 3D case, pair of phase steps). Thus, it is possible, for example, to record the corresponding data associated with the individual frequencies in the k space for each individual phase step of the plurality of phase steps of the at least one additional dimension or in the 3D case for each pair of phase steps, so that, for example, a set of data of a two-dimensional or three-dimensional k space can be recorded.

Steps 610 through bis 660 of method 600 comprise the selecting of a a subset of data from the first set of data U₁^{α} as well as the selecting of a subset of data from the second set of data U₂^{α}.

In step 610, a phase step pₛ with s∈{1, . . . , w} of the plurality of w phase steps p₁, . . . , p_{w} is selected. In the exemplary 3D case, this step 610 includes selecting a pair of phase steps p_{s,q} with s∈{1, . . . , w}, wherein the phase step pₛ represents, for example, the selected first phase step of the first plurality of w phase steps p₁, . . . , p_{w} and a selected phase step p′_{q} represents the selected second phase step of the second plurality of w₂ phase steps p′₂, . . . , p′_{w}_{₂} by which the pair of phase steps p_{s,q} comprising the first phase step pₛ and the second phase step p′_{q} can be defined.

Thereafter, a frequency fⱼ with j∈{1, . . . , v} of the plurality of v frequencies f₁, . . . , fᵥ is selected in step 620. The frequency fⱼ can, for example, be associated with a recording index which is associated with the corresponding data value from the plurality of data values associated in the k space with the selected phase step or the selected pair of phase steps.

Based on the selection of the the phase steps pₛ (or the selection of the pair of phase steps p_{s,q}) and the selection of the frequency fⱼ, a data value u_{2,l}^{α} associated with this phase step pₛ (or the pair of phase steps p_{s,q}) and this frequency fⱼ in the k space can be selected from the second set of data U₂^{α}. In this, the phase step pₛ (or pair of phase steps p_{s,q}) selected in step 610, and the frequency fⱼ selected in step 620 are selected such that the data value u_{2,l}^{α} associated with this phase step pₛ (or this pair of phase steps p_{s,q}) and this frequency fⱼ in the k space lies in the time sub-segment of the second time segment for which the value of a relative contrast agent enhancement is to be determined, wherein the index l indicates the corresponding point in the k space as a function of fⱼ and pₛ (or p_{s,q}).

For example, the point in time of the recording T(s) can be determined at the start of each phase step pₛ of a recording of an nth set of data Uₙ^{α} as follows:

T(s,n)=(s−1)T_{R}+T_{E}+tₙ

Thus, T_{R} represents, for example, the repetition time between two adjacent phase steps, T_{E} represents, for example, the time after which the antennae will start recording, and tₙ represents, for example, the point in time when the first phase step s=1 of the nth set of data Uₙ^{α}starts. In the 3D case, the point in time of the recording T(s,q,n) can, for example, be determined as follows:

T(s,q,n)=T′(T_{R},s,q)+T_{E}+tₙ,

wherein T′(T_{R},s,q) can represent the summed up time of all repetition times for all combinations of pairs of phase steps run through in the MRI recording prior to the pair p_{s,q} having been entered.

Depending on the type of recording, the time of recording of the data values for the various frequencies during a phase step may be negligibly short, so that the recorded data values for this one phase step pₛ and the various frequencies can be associated at least approximately or even exactly with the same point in time T(s).

If, for example, between the individual frequencies there is a time segment that is significant as impulse response for the acquisition of a frequency spectrum, which time segment is defined, for example, by T_{f}, the point in time for a certain phase step pₛ and a certain frequency step fⱼ can be determined, for example, as follows:

T(s,j,n)=(s−1)T_{R}+(j−1)T_{f}+T_{E}+tₙ,

or for a certain pair of phase steps p_{s,q} it can be determined as follows:

T(s,q,j,n)=T′(T_{R},s,q)+(j−1)T_{f}+T_{E}+tₙ

The data value u_{2,l}^{α} associated in the k space with the selected phase step pₛ (or the selected pair of phase steps p_{s,q}) and the selected frequency fⱼ is associated with the subset of data from the second set of data in step 630, for example by using the indices i_{x∈{}1, . . . , m} described above, wherein for example a counter increments the variable x of the index iₓ from a starting value, such as, for example, 0 or 1, for each run through of the loop through step 630 in method 600. Thus, in step 630, the index iₓ can be set to the index l=f(pₛ, fⱼ) selected in accordance with the selected phase step pₛ and the selected frequency fⱼ, or to the index l=f(p_{s,q}, fⱼ) of the k space associated with the selected pair of phase steps p_{s,q} and the selected frequency fⱼ:

iₓ=l.

Thus, for example, u_{2,i}_{ₓ}^{α} corresponds to the xth data value of the subset of data from the second set of data selected in the xth run-through of the loop in step 630 of method 600 associated and associated with the subset of data from the second set of data.

Similarly to the procedure in step 630, it is possible, based on the selection of the phase step pₛ (or the pair of phase steps p_{s,q}) and the selection of the frequency step fⱼ, to select in step 640 a data value u_{1,l}^{α} associated with this phase step pₛ (or this pair of phase steps p_{s,q}) and this frequency fⱼ in the k space from the first set of data U₁^{α}. As the phase step pₛ (or the pair of phase steps p_{s,q}) and the frequency fⱼ is equal to the phase step pₛ (or pair of phase steps p_{s,q}) and frequency fⱼ underlying step 630, the index l already selected step 630 can be to select the data value u_{1,l}^{α}, because the data value u_{2,l}^{α} of the second set of data selected in step 630 lies in the same point in the k space as the data value u_{1,l}^{α} of the first set of data selected in step 640. The data value u_{1,l}^{α} from the first set of data U₁^{α} thus selected therefore lies automatically in the time sub-segment of the first time segment.

For example, the data value u_{1,l}^{α} associated with the subset of data from the first set of data can be expressed through u_{1,i}_{ₓ}^{α} using the indices iₓ=l defined in step 630.

Thus, a data value u_{2,i}_{ₓ}^{α} the second set of data and a data value u_{2,i}_{ₓ}^{α} of the first set of data will each lie in step 630 and step 640, which data values, by selecting the phase step or of the pair of phase steps in step 610 and the frequency in step 620 accordingly, lie in the time sub-segment of the second time segment or of the first time segment.

The order of steps 630 and 640 can be switched, the two steps 630 and 640 can also be performed simultaneously.

Step 650 allows to verify whether a further frequency in the k space is to be used to determine the value of a relative contrast agent enhancement. If so, the method jumps back to step 620 and selects this further frequency as new frequency.

Accordingly, in step 630, a data value u_{2,l}^{α} associated in the k space with a phase step pₛ (or pair of phase steps p_{s,q}) already selected earlier in step 610 and with the frequency fⱼ newly selected in step 620, is selected from the second set of data, wherein the index l indicates the corresponding point in the k space as a function of fⱼ and pₛ. For example, the counter x of the index iₓ can be incremented and set to iₓ=l.

Analogously, a data value u_{1,i}^{α} associated in the k space with a phase step pₛ (or pair of phase steps p_{s,q}) already selected earlier in step 610 and with the frequency fⱼ newly selected in step 620, is selected from the first set of data, wherein this data value can be express by u_{1,i}_{ₓ}^{α}.

Thus, in step 630, the data value u_{2,l}^{α} selected in step 630 can be added to the subset of data from the second set of data, and in step 640, the data value u_{1,i}_{ₓ}^{α} selected in step 640 can be added to the subset of data from the first set of data.

For example, it is possible to run through the loop between steps 620 and 650 until all frequencies of the plurality of frequencies have been selected in step 620, as a result of which, for a phase step (or a pair of phase steps) selected in step 610, the data of the subset of data from the second set of data in the first dimension is associated with all frequencies, and in analogy hereto, the data of the subset of data from the first set of data in the first dimension is likewise associated with all frequencies. It is also possible, for example, to only select a subset of several frequencies from the plurality of frequencies through the loop between steps 620 and 650, or this loop can also not be run through at all or only once, so that only one single frequency is selected and thus, for the phase step (or pair of phase steps) selected in step 610, the data of the subset of data from the second set of data in the first dimension is associated only with this single frequency, with step 650 being able to be omitted.

If it is determined in step 650 that no further frequency needs to be selected, it is possible to check in the optional step 660 whether a yet another phase step (or pair of phase steps) needs to be selected.

If this is the case, i.e. if the data of the subset of data from the second set of data and, accordingly, the data of the subset of data from the first set of data are yet to be associated with at least on further phase step of the plurality of phase steps, the method jumps back to step 610 and selects this phase step (or a corresponding, new pair of phase steps) there, and the method continues in step 620 with the selection of a frequency. For example, the loop between steps 620 and 650 can select the exact same at least one frequency, or subset of frequencies from the plurality of frequencies, or all frequencies of the plurality of frequencies.

Thus, method 600 can be performed, for example, for a subset of phase steps of the plurality of phase steps of an additional dimension, wherein the subset of phase steps preferably includes directly successive phase steps, such as, for example, in the MRI-recording in this additional dimension, phase steps that directly succeed each other in time. For example, in the 3D case, the second phase step p′_{q} can be kept constant or the same selection can be made, and the phase step pₛ newly selected in step 610 is selected form the subset of phase steps of the plurality of phase steps of the one additional dimension, so that as a result of the selection of the respective new phase step pₛ in step 610, new pairs of phase steps p_{s,q} are selected in each case.

Thus, one phase step of the first plurality of phase steps for phase encoding in the first additional dimension and one phase step of the second plurality of phase steps for phase encoding in the second additional dimension are selected in the 3D scan, wherein this selected phase step in the first additional dimension and the respectively selected phase step in the second additional dimension can be joined as a respective pair of phase steps, wherein the phase encoding in the first additional dimension and the phase encoding in the second additional dimension can be done simultaneously in accordance with the respective pair of phase steps. This can be implemented, for example, by applying, in the spatial direction of the first additional dimension, a gradient field in accordance with the phase encoding of the first additional dimension and, simultaneously, in the spatial direction of the second additional dimension, a gradient field in accordance with the phase encoding of the second additional dimension, and switching these gradient fields off again prior to the reading of the received signal.

The external loop of method 610, i.e. the optional checking in step 660 of whether a further phase step 660 (or pair of phase steps) needs to be selected, can, however, also be omitted and is therefore be considered only as optional, so that method 600, for example, selects only exactly one single phase step (or one single pair of phase steps) in step 610, and then jumps directly from step 650 to determining the value of a relative contrast agent enhancement associated with the time sub-segment of the second time segment.

Thus, with method 600, the subset of data of the second set of data can be selected, wherein upon running through step 630 for the kth time, a total of k data values u_{2,i}_{₁}^{α}, . . . , u_{2,i}_{ₖ}^{α} of the second set of data U₂^{α} with 1≦k<m represent the selected data values of the subset of data of the second set of data, and with method 600, the subset of data of the second set of data can be selected, wherein upon running through step 640 for the kth time, a total of k data values u_{1,i}_{₁}^{α}, . . . , u_{1,i}_{ₖ}^{α} of the second set of data U₁^{α} with 1≦k<m represent the selected data values of the subset of data of the first set of data.

Based on this selected subset of data of the first set of data and on this selected subset of data of the second set of data, it is possible in step 670 to determine a value of a relative contrast agent enhancement associated with the time sub-segment wherein this step can be performed, for example, as described in step 230 of FIG. 2 and in all previously described methods.

In this context, it is important that for each of the phase steps (or for each pair of phase steps) all spins located in S_{E} contribute to the recorded signal. Each entry in the recorded k space thus contains information from the entire area, and not only from spatially localized parts of S_{E}.

If the time sub-segment of the second time segment is to be a relatively short time segment, i.e. if the temporal resolution of the value of a relative contrast agent enhancement determined in step 670 is to be relatively small, it is possible in the method 600 to preferably select only a single phase step (or only a single pair of phase steps), so that all data values of the subset of data of the first set of data and of the subset of data of the second set of data are associated solely with this one selected phase step (or sole pair of phase steps).

If, therefore, the data values of the subset of data of the second set of data are associated with exactly one phase step pₛ (or exactly one pair of phase steps p_{s,q}), the corresponding time sub-segment of the second time segment lies, for example, in the time segment between T(s,n) and T(s+1,n), and if the frequencies run through during the recording for this phase step pₛ (or exactly one pair of phase steps p_{sq}) are run through in a very short time, the time sub-segment can approximately be seen as a very short time segment which starts at T(s,n) and ends very shortly thereafter, i.e. the time sub-segment of the second time segment can be considered an approximate point in time.

If the time sub-segment of the second time segment is to represent, for example, a somewhat longer time segment, i.e. if the temporal resolution of the value determined in step 670 of a relative contrast agent enhancement is to be higher than in the aforementioned example, in which only one single phase step (or a single pair of phase steps) is selected, it is possible in method 600 to preferably select several phase steps of the plurality of phase steps (or several pairs of phase steps), wherein these several phase steps (or several pairs of phase steps) are preferably directly adjacent, so that the selected subset of data of the first set of data and the subset of data of the second set of data are associated with these several selected phase steps (or several selected pairs of phase steps).

In order to determine a value of a relative contrast agent enhancement associated with the time sub-segment of the second time segment, the method can, for example, contain an optional step (not shown in FIG. 6) between step 640 and step 650, in which the differential value between the data value u₂_{ₓ}^{α} of the second set of data selected immediately prior in step 630, and the data value u_{1,i}_{ₓ}^{α} of the first set of data selected immediately prior in step 640 is calculated as follows:

dᵢ_{ₓ}^{α}=u_{2,i}_{ₓ}^{α}−u_{1,i}_{ₓ}^{α}

It is also possible for these differential values to be determined, for example, only after running through the loop or loops of method 600, for example as described above.

Then, the calculated differential values dᵢ_{₁}^{α}, . . . , dᵢ_{ₖ}^{α} can be used, for example, as basis for the calculation of at least one metric in the method 300 for determining a value of a relative contrast agent enhancement shown in FIG. 3, any of the other methods described above.

However, it is also possible, for example, to calculate the differential values between the data values of the second set of data and the data values of the first set of data in advance.

Thus, for example, all differential values Dhu α between the data values of the data of the second set of data and the data values of the data of the first set of data can be formed in advance, for example by

D_{α}=U₂^{α}U₁^{α},

with D^{α}=(d₁^{α}, . . . , dₘ^{α}) and for example

dₗ^{α}=u_{2,l}^{α}−u_{1,l}^{α}

with l∈{1, . . . , m}.

However, it is also possible to only form subsets of differential values between a subset of data values of the data of the second set of data and a corresponding subset of data values of the data of the first set of data in advance.

FIG. 6b shows an example of a method 600′ according to a sixth embodiment, which is based on the example of a method 600 shown in FIG. 6a according to a fifth embodiment. Thus, the explanations provided with regard to method 600 can be, where possible, carried over to the method 600′ according to a sixth embodiment, wherein this applies in particular to steps 610, 620, 650 (optional) and 660 (optional).

In step 635, a differential value dᵢ_{ₓ}^{α}is determined for the selected phase step (or the selected pair of phase steps) and for the selected frequency, which represents a difference between a data value u_{2,i}_{ₓ}^{α} of the second set of data associated with the selected phase step (or the selected pair of phase steps) and the selected frequency, and the data value u_{1,i}_{ₓ}^{α} of the first set of data associated with the selected phase step (or selected pair of phase steps) and the selected frequency, where the differential value dᵢ_{ₓ}^{α} can be defined, for example, as follows:

dᵢ_{ₓ}^{α}=u_{2,i}_{ₓ}^{α}−u_{1,i}_{ₓ}^{α}

The differential value dᵢ_{ₓ}^{α} is thus calculated based on a data value u_{2,i}_{ₓ}^{α} of the second set of data associated with the selected phase step (or the selected pair of phase steps) and the selected frequency, wherein this associated data value u_{2,i}_{ₓ}^{α} represents part of the subset of data from a second set of data, and based on a data value u_{1,i}_{ₓ}^{α} of the first set of data associated with the selected phase step (or the selected pair of phase steps) and the selected frequency, wherein this associated data value u_{1,i}_{ₓ}^{α} represents part of the subset of data from a first set of data. Thus, step 635 comprises, for example, at least implicitly, the selecting of a data value u_{2,i}_{ₓ}^{α} of the second set of data associated with the selected phase step (or the selected pair of phase steps) and the selected frequency (in accordance with step 630 from FIG. 6a), and the selecting of a data value u₁ₘᵢ_{ₓ}^{α} of the first set of data associated with the selected phase step (or the selected pair of phase steps) and the selected frequency (in accordance with step 640 from FIG. 6a), as the differential value dᵢ_{ₓ}^{α} calculated in step 635 is formed from these selected data values u_{2,i}_{ₓ}^{α} and u_{1,i}_{ₓ}^{α}.

The differential value dᵢ_{ₓ}^{α} can be calculated in step 635, but it can also be determined from the already calculated differential values described above. For this, for example, the index iₓ will be set to the index l=f(pₛ, fⱼ) (or in the 3D case l=f(p_{s,q}, fⱼ)) of the k space associated accordingly with the selected phase step pₛ (or the selected pair of phase steps p_{s,q}) and the selected frequency fⱼ:

iₓ=f(Pₛ, fⱼ), or

iₓ=f(p_{s,q}, fⱼ),

and the corresponding differential value dᵢ_{ₓ}^{α} is determined from the differential values calculated in advance.

In step 670′ of the method 600′, a value of a relative contrast agent enhancement associated with the time sub-segment of the second time segment is determined based on the differential values in step 635 or in steps 635, wherein this step can be carried out, for example, as described in step 230 of FIG. 2 and all previously described methods.

With regard to the methods 600 and 600′, the phase steps (or pairs of phase steps) and frequencies selected in steps 610 and 620 are selected, for example, such that, the corresponding point in the k space (for example, two- or three-dimensional), which can be expressed, for example, through the index iₓ with iₓ=f(pₛ, fⱼ) (or iₓ=f(p_{s,q}, fⱼ)), lies, with regard to the recorded second set of data, in the time sub-segment of the second time segment. Thus, only data values from the second set of data recorded in this time sub-segment are used to determine the value of a relative contrast agent enhancement associated with this time sub-segment in step 670 or in step 670′.

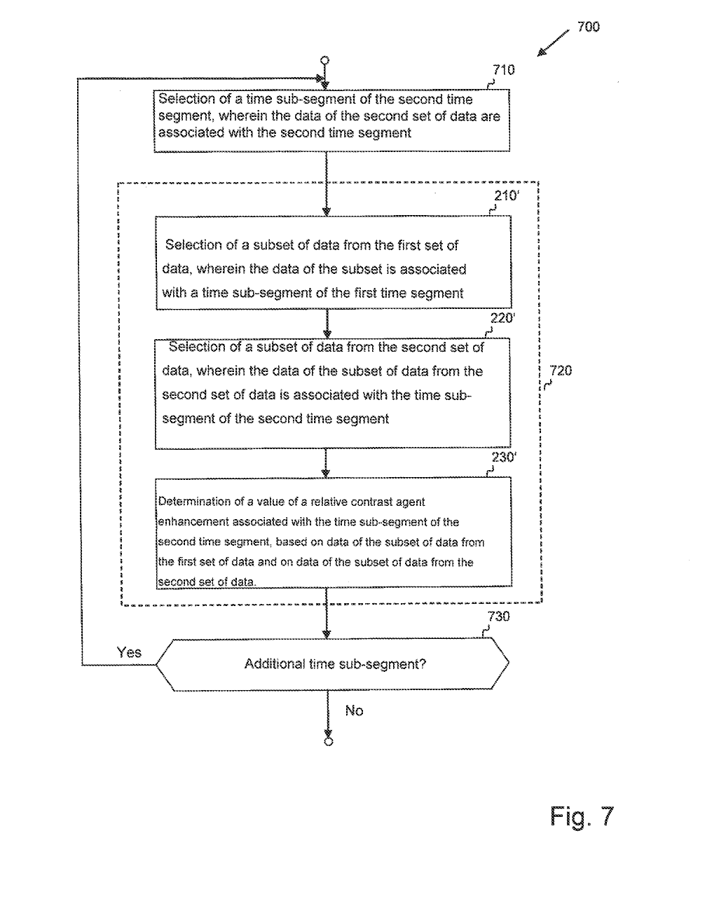

FIG. 7 shows an example of a method according to a seventh embodiment.

This example of method 700 according to a seventh embodiment can be used to determine a plurality of values of a relative contrast agent enhancement, wherein each of the values of a relative contrast agent enhancement is associated with a different time sub-segment of the second time segment.

In step 710, a time sub-segment from the second time segment is selected.

Thereafter, in step 720, shown in FIG. 7 as a dotted line, a value of a relative contrast agent enhancement associated with this time sub-segment of the second time segment is determined based on data of a selected subset of data from the first set of data and based on data of a selected subset of data from the second set of data. This step 720 can be performed by each of the methods described above, such as, for example, by the method shown in FIG. 2, wherein step 210′ in FIG. 7 corresponds to step 210 in FIG. 2, step 220′ in FIG. 7 corresponds to step 220 in FIG. 2, and step 230′ in FIG. 7 corresponds to step 230 in FIG. 2, or step 720 can be realized, for example, through steps 600 to 670 of the method 600 or 600′ shown in FIG. 6a or, for example, through steps 610 to 670′ of the method 600 or 600′ shown in FIG. 7a.

In step 730 it is verified whether there is a further time sub-segment of the second time segment for which a value of a relative contrast agent enhancement needs to be determined. For example, it is possible to verify if for a time sub-segment of the plurality of time sub-segments, no value of a relative contrast agent enhancement has been determined yet. If so, the method jumps back to step 710 and selects this further time sub-segment in step 710, so that the value of a relative contrast agent enhancement associated with this newly selected time sub-segment is determined subsequently in step 720.

If it is determined in step 730 that no further time sub-segment of the second time segment exists, for which a relative contrast agent enhancement needs to be determined, the method can, for example, end or, optionally, or further process the determined values of a relative contrast agent enhancement.

The second time segment in a plurality of time sub-segments can, for example, be subdivided, wherein a corresponding value of a relative contrast agent enhancement is determined for each of the time sub-segments. The plurality of time sub-segments of the second time segment can, for example, also represent a selection of time sub-segments of the second time segment, so that values of relative contrast agent enhancements are determined only for especially selected time sub-segments that do not cover the entire second time segment.

The time sub-segments of the plurality of time sub-segments can, for example, be selected such that at least one time sub-segment of the plurality of time sub-segments is associated, in each case, with exactly one phase step from the plurality of phase steps (or exactly one pair of phase steps), which is then selected, for example, in step 610 of method 600 or 600′, and/or that at least one time sub-segment of the plurality of time sub-segments is associated, in each case, with several, preferably adjacent phase steps from the plurality of phase steps (or exactly one pair of phase steps).

The time sub-segments can, in each case, have the same length, but they can also have different lengths.

According to an advantageous embodiment, it is suggested that the method comprise determining a value of an absolute contrast agent enhancement based on the determined value of a relative contrast agent enhancement and a lookup database or a mapping function.

This lookup database can, for example, map a non-linear dependence of the values of an absolute contrast agent enhancement on the values of the relative contrast agent enhancement. This non-linear dependence can, for example, be calculated by way of a calibration method using calibration phantoms.

1. Method, performed by at least one apparatus, comprising:
Calculation of a value of a relative contrast agent enhancement associated with a time sub-segment of a second time segment based on data of a subset of data from a first set of data and based on data of a subset of data from a second set of data,
wherein the first set of data comprises data of a multi-dimensional k space of a magnetic resonance scan recorded without the influence of a contrast agent and was recorded in a first time segment,
wherein the second set of data comprises data of a multi-dimensional k space of a magnetic resonance scan recorded with the influence of a contrast agent and was recorded in the second time segment, which is different from the first time segment,
wherein the data of the subset of data from the second set of data is associated with time sub-segment of the second time segment, and the data of the subset of data from the first set of data is essentially associated with the same region in the k space as the data of the subset of data from the second set of data,
wherein the data of the subset of data from the first set of data comprises several data values and the data of the subset of data from the second set of data comprises several data values.

2. Method according to claim 1, wherein the multi-dimensional k space be frequency-encoded in a first dimension with a plurality of frequencies and phase-coded in at least one additional dimension with a plurality of phase steps.

3. Method according to claim 2, wherein the time sub-segment of the second time segment is such that the data of the subset of data from the second set of data in one dimension of the at least one additional dimension is associated with one of the following:
exactly one phase step of the plurality of phase steps of the dimension, and
several successive phase steps of the plurality of phase steps of the dimension.

4. Method according to claim 3, wherein the time sub-segment of the second time segment is determined based on the acquisition parameters of the exactly one phase step or the several successive phase steps, the echo time and the repetition time.

5. Method according to claim 3, wherein the data of the subset of data from the second set of data in the first dimension is associated with one of the following:
exactly one frequency of the plurality of frequencies,
a subset of several frequencies from the plurality of frequencies, and
all frequencies of the plurality of frequencies.

6. Method according to claim 1 wherein the value of a relative contrast agent enhancement associated with the time sub-segment of the second time segment be determined based on the differential values between the data of the subset of the second set of data and the data of the subset of the first set of data, wherein a respective differential value of the differential values is formed based on the difference between one data value of the data of the subset of the second subset and the data value of the data of the subset of the first set of data associated with said data value.

7. Method according to claim 6, wherein the method includes calculating a metric based on the determined differential values, wherein the metric represents a measure for the deviation between the differential values and a value of a relative contrast agent enhancement.

8. Method according to claim 7, wherein calculating the metric for each of the differential values includes calculating a differential value of the metric, which represents the difference between the respective differential value and a value of a relative contrast agent enhancement, and calculating the metric includes calculating the deviation measure on the basis of the calculated differential values of the metric.

9. Method according to claim 6, wherein the method includes calculating a plurality of metrics, wherein each of the plurality of metrics is associated with a different value of a relative contrast agent enhancement, and wherein the method includes the selection of that value of a relative contrast agent enhancement which is associated with the metric with the smallest deviation measure.

10. Method according to claim 7, wherein the metric includes a filter function, wherein the filter function is configured to frequency-selectively weight data from the first subset of data from the first set of data and data from the subset of data from the second set of data such that at least some data associated with higher frequencies has less influence on the metric than data associated with lower frequencies.

11. Method according to claim 7, wherein based on at least one sub-image space in the image space, a sub-image mask associated with the k space is defined in the k space by transforming the at least one sub-image space in the k space, wherein determining a value of a relative contrast agent enhancement associated with the time sub-segment of the second time segment based on data of the subset of data from the first set of data and data of the subset of data from the second set of data and further based on the sub-image space mask associated with the k space.

12. Method according to claim 1, wherein the method includes selecting the at least one sub-image space in the image space based on a representation of the first or of the second set of data in the image space.

13. Method according to claim 1, wherein the method includes determining a value of an absolute contrast agent enhancement based on the determined value of a relative contrast agent enhancement and a lookup database or a mapping function.

14. Method according to claim 1, wherein the method includes determining a plurality of values of a relative contrast agent enhancement for a plurality of time sub-segments of the second time segment based on the data of the subset of data from the first set of data and the data of the subset of data from the second set of data, wherein each of the values of a relative contrast agent enhancement is associated with a different time sub-segment of the plurality of time sub-segments.

15. (canceled)

16. Computer-readable medium comprising:
program code for performing the method according to claim 1, when said program is executed on a processor.

17. (canceled)

18. Apparatus comprising at least one processor, at least one storage medium containing a computer program code, wherein the at least one storage medium and the computer program code are designed to carry out the method according to claim 1 together with the at least one processor.

19. A computer readable storage medium in which computer program code is stored, the computer program code causing an apparatus to perform the following when executed by a processor:
Calculation of a value of a relative contrast agent enhancement associated with a time sub-segment of a second time segment based on data of a subset of data from a first set of data and based on data of a subset of data from a second set of data,
wherein the first set of data comprises data of a multi-dimensional k space of a magnetic resonance scan recorded without the influence of a contrast agent and was recorded in a first time segment,
wherein the second set of data comprises data of a multi-dimensional k space of a magnetic resonance scan recorded with the influence of a contrast agent and was recorded in the second time segment, which is different from the first time segment,
wherein the data of the subset of data from the second set of data is associated with time sub-segment of the second time segment, and the data of the subset of data from the first set of data is essentially associated with the same region in the k space as the data of the subset of data from the second set of data,
wherein the data of the subset of data from the first set of data comprises several data values and the data of the subset of data from the second set of data comprises several data values.
